(19) United States
(12) Patent Application Publication
(10) Pub. No.: US 2017/0347916 A1
(43) Pub. Date: Dec. 7, 2017

(54) Manipulation Support Apparatus, Insert System, and Manipulation Support Method

(71) Applicant: Olympus Corporation, Tokyo (JP)

(72) Inventors: Jun Hane, Tokyo (JP); Eiji Yamamoto, Tokyo (JP)

(73) Assignee: Olympus Corporation, Tokyo (JP)

(21) Appl. No.: 15/684,242

(22) Filed: Aug. 23, 2017

Related U.S. Application Data
(63) Continuation of application No. PCT/JP2015/055932, filed on Feb. 27, 2015.

(51) Int. Cl. A61B 5/06; A61B 1/00; A61B 1/307; A61B 1/04; A61B 34/20

(52) U.S. Cl. CPC A61B 5/065; A61B 1/307; A61B 1/04; A61B 1/00045; A61B 1/00039; A61B 2034/2061

(57) ABSTRACT

Example embodiments of the present invention relate to a manipulation support apparatus. The apparatus may include a processor and memory storing instructions that when executed on the processor cause the processor to perform the operation of acquiring detection data from a sensor provided in an inserted object which is inserted into a subject body. The detection data may be associated with a state of the inserted object. The apparatus then may decide setting information based on at least one of inserted object information and the user information. The apparatus then may generate support information for a manipulation of the inserted object based on the detection data and the setting information.

### CROSS-REFERENCE TO RELATED APPLICATION(S)

This application is a continuation application of PCT Application No. PCT/JP2015/055932 filed Feb. 27, 2015, which is hereby incorporated by reference in its entirety.

### TECHNICAL FIELD

The present invention relates to a manipulation support device, an insert system, and a manipulation support method.

### BACKGROUND

In general, there has been known an insertion-extraction device including an insert having an elongated shape, such as an insertion portion of an endoscope. For example, if a user is able to perform manipulation while recognizing a state of the insertion portion during insertion of the insertion portion of the endoscope into a subject, it is easy for the user to insert the insertion portion into the subject. Therefore, there has been known technology for recognition of the state of the insert of the insertion-extraction device.

For example, a conventional insertion portion of an endoscope may be provided with an endoscope inserting shape detection probe. The endoscope inserting shape detection probe includes detecting-light transmitting means. The detecting-light transmitting means has a configuration in which a light loss amount varies depending on a bending angle. A use of the endoscope inserting shape detection probe allows the bending angle of the insertion portion of the endoscope to be detected. As a result, it is possible to form a bending shape of the endoscope insertion portion, again.

Another conventional endoscope insertion portion may be provided with a sensor support and a strain gauge is installed on the sensor support. A use of the strain gauge allows an external force applied to the endoscope insertion portion in a specific direction to be detected. As a result, it is possible to achieve information associated with the external force applied to the endoscope insertion portion.

Another conventional endoscope system may be provided with shape estimation means that estimates a shape of an endoscope insertion portion. In the endoscope system, a warning is issued as necessary, based on the shape of the endoscope insertion portion estimated by the shape estimation means. For example, when the endoscope insertion portion is detected to have a loop shape, a warning for calling attention is issued as a display or a sound.

A device or a method for achieving further detailed recognition of the state of the insertion portion of the insertion-extraction device is further demanded to be provided. Further, a device or a method that is capable of providing useful support information for a manipulator in manipulation of the insertion portion, based on the state of the insertion portion, is demanded to be provided.

### SUMMARY

Example embodiments of the present invention relate to a manipulation support apparatus. In one aspect, the manipulation support apparatus comprises a processor, and memory storing instructions that when executed on the processor cause the processor to perform the operations of acquiring detection data from a sensor provided in an inserted object which is inserted into a subject body, the detection data being associated with a state of the inserted object, deciding setting information based on at least one of inserted object information associated with at least one of the inserted object and the sensor and user information associated with at least one of a manipulator who manipulates the inserted object and an operation performed by using the subject body and the inserted object and generating support information for a manipulation of the inserted object based on the detection data and the setting information.

### BRIEF DESCRIPTION OF DRAWINGS

Objects, features, and advantages of embodiments disclosed herein may be better understood by referring to the following description in conjunction with the accompanying drawings. The drawings are not meant to limit the scope of the claims included herewith. For clarity, not every element may be labeled in every Figure. The drawings are not necessarily to scale, emphasis instead being placed upon illustrating embodiments, principles, and concepts. Thus, features and advantages of the present disclosure will become more apparent from the following detailed description of exemplary embodiments thereof taken in conjunction with the accompanying drawings in which:

FIG. 1 is a diagram schematically illustrating an example of a configuration of an insertion-extraction device according to an embodiment.

FIG. 2 is a diagram illustrating an example of a configuration of a sensor provided in an endoscope according to the embodiment.

FIG. 3 is a diagram illustrating another example of a configuration of the sensor provided in the endoscope according to the embodiment.

FIG. 4 is a diagram illustrating still another example of a configuration of the sensor provided in the endoscope according to the embodiment.

FIG. 5 is a diagram schematically illustrating an example of a configuration of a shape sensor according to the embodiment.

FIG. 6 is a diagram schematically illustrating an example of a configuration of an inserting amount sensor according to the embodiment.

FIG. 7 is a diagram schematically illustrating another example of a configuration of the inserting amount sensor according to the embodiment.

FIG. 8 is a diagram for describing information that is obtained by the sensor according to the embodiment.

FIG. 9 is a diagram for describing a first state determination method and schematically illustrating a state of movement of an insertion portion between a time point t1 and a time point t2.

FIG. 10 is a diagram for describing the first state determination method and schematically illustrating an example of a state of movement of the insertion portion between the time point t2 and a time point t3.

FIG. 11 is a diagram for describing the first state determination method and schematically illustrating another example of the state of the movement of the insertion portion between the time point t2 and the time point t3.

FIG. 12 is a block diagram schematically illustrating an example of a configuration of an insertion-extraction support device that is used in the first state determination method.

FIG. 13 is a flowchart illustrating an example of a process in the first state determination method.

FIG. 14 is a diagram for describing a first modification example of the first state determination method and schematically illustrating the state of the movement of the insertion portion between the time point t1 and the time point t2.

FIG. 15 is a diagram for describing the first modification example of the first state determination method and schematically illustrating an example of the state of the movement of the insertion portion between the time point t2 and the time point t3.

FIG. 16 is a diagram for describing the first modification example of the first state determination method and schematically illustrating another example of the state of the movement of the insertion portion between the time point t2 and the time point t3.

FIG. 17 is a diagram for describing a second modification example of the first state determination method and schematically illustrating an example of the state of the movement of the insertion portion.

FIG. 18 is a diagram for describing a second state determination method and schematically illustrating the state of movement of the insertion portion between the time point t1 and the time point t2.

FIG. 19 is a diagram for describing the second state determination method and schematically illustrating an example of the state of the movement of the insertion portion between the time point t2 and the time point t3.

FIG. 20 is a diagram for describing the second state determination method and schematically illustrating another example of the state of the movement of the insertion portion between the time point t2 and the time point t3.

FIG. 21 is a graph illustrating an example of a change in a position of an attention point obtained as time elapses.

FIG. 22 is a block diagram schematically illustrating an example of a configuration of the insertion-extraction support device that is used in the second state determination method.

FIG. 23 is a flowchart illustrating an example of a process in the second state determination method.

FIG. 24 is a diagram for describing a modification example of the second state determination method and schematically illustrating an example of the state of the movement of the insertion portion.

FIG. 25 is a diagram for describing the modification example of the second state determination method and schematically illustrating another example of the state of the movement of the insertion portion.

FIG. 26 is a diagram for describing a third state determination method and schematically illustrating the state of the movement of the insertion portion between the time point t1 and the time point t2.

FIG. 27 is a diagram for describing the third state determination method and schematically illustrating an example of the state of the movement of the insertion portion between the time point t2 and the time point t3.

FIG. 28 is a diagram for describing the third state determination method and schematically illustrating another example of the state of the movement of the insertion portion between the time point t2 and the time point t3.

FIG. 29 is a diagram for describing the third state determination method and schematically illustrating an example of the state of the movement of the insertion portion.

FIG. 30 is a diagram for describing the third state determination method and schematically illustrating another example of the state of the movement of the insertion portion.

FIG. 31 is a diagram schematically illustrating a change in the position of the attention point on the insertion portion.

FIG. 32 is a diagram schematically illustrating an example of the state of the movement of the insertion portion.

FIG. 33 is a graph illustrating an example of a change in a distance from a front end of the insertion portion to the attention point obtained as time elapses.

FIG. 34 is a diagram schematically illustrating another example of the state of the movement of the insertion portion.

FIG. 35 is a graph illustrating another example of the distance from the front end of the insertion portion to the attention point obtained as time elapses.

FIG. 36 is a graph illustrating an example of a change in a self-compliance property obtained as time elapses.

FIG. 37 is a block diagram schematically illustrating an example of a configuration of the insertion-extraction support device that is used in the third state determination method.

FIG. 38 is a flowchart illustrating an example of a process in the third state determination method.

FIG. 39 is a diagram for describing a fourth state determination method and schematically illustrating an example of the state of the movement of the insertion portion.

FIG. 40 is a diagram for describing a relationship between a tangential direction and a moving amount in the fourth state determination method.

FIG. 41 is a graph illustrating an example of a change in a ratio between displacements of the insertion portion in the tangential direction obtained as time elapses.

FIG. 42 is a graph illustrating another example of a change in the ratio between the displacements of the insertion portion in the tangential direction obtained as time elapses.

FIG. 43 is a graph illustrating an example of a change in sideway movement of the insertion portion obtained as time elapses.

FIG. 44 is a block diagram schematically illustrating an example of a configuration of the insertion-extraction support device that is used in the fourth state determination method.

FIG. 45 is a flowchart illustrating an example of a process in the fourth state determination method.

FIG. 46 is a diagram for describing a modification example of the fourth state determination method and schematically illustrating an example of the state of the movement of the insertion portion.

FIG. 47 is a graph illustrating an example of a change in front end advance of the insertion portion obtained as time elapses.

FIG. 48 is a diagram schematically illustrating an example of a configuration of a manipulation support information generating device according to the embodiment.

FIG. 49 is a diagram illustrating an example of a menu item associated with inputting of first manipulator information.

FIG. 50 illustrates an example of an image as manipulation support information that is displayed on a display device.

FIG. 51 illustrates another example of the image as the manipulation support information that is displayed on the display device.

FIG. 52 is a diagram illustrating another example of the menu item associated with the inputting of the first manipulator information.

FIG. 53 is a diagram illustrating an example of user specific information as an example of second manipulator information.

FIG. 54 is a diagram illustrating an example of subject information as an example of the second manipulator information.

FIG. 55 is a diagram illustrating an example of information associated with setting criteria as an example of the second manipulator information.

FIG. 56 is a diagram illustrating an example of device information as an example of the second manipulator information.

FIG. 57 is a diagram for describing an example of generation of the manipulation support information.

FIG. 58 is a diagram schematically illustrating an example of a configuration employed in a case where a plurality of inserts is used in the insertion-extraction device.

### DETAILED DESCRIPTION

According to the present invention, it is possible to provide support information associated with manipulation of an insert.

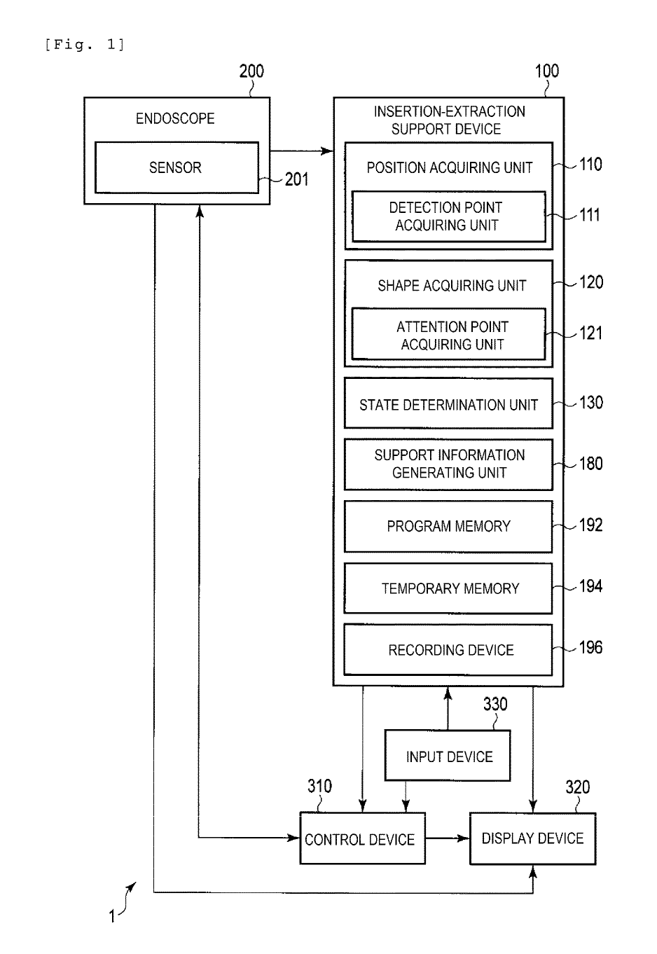

An embodiment of the invention will be described with reference to the figures. FIG. 1 is a diagram schematically illustrating an example of a configuration of an insertion-extraction device 1 according to the embodiment. The insertion-extraction device 1 includes an insertion-extraction support device 100, an endoscope 200, a control device 310, a display device 320, and an input device 330.

The endoscope 200 is a common endoscope. The control device 310 is a control device that controls an operation of the endoscope 200. The control device 310 may acquire, from the endoscope 200, information necessary for control. The display device 320 is a common display device. The display device 320 includes, for example, a liquid crystal display. The display device 320 displays an image acquired by the endoscope 200 or information associated with the operation of the endoscope 200, which is generated in the control device 310. The input device 330 receives an input of a user to the insertion-extraction support device 100 and the control device 310. For example, the input device 330 includes a button switch, a dial, a touch panel, a keyboard, or the like. The insertion-extraction support device 100 performs information processing for supporting insertion or extraction of the insertion portion of the endoscope 200 into or from a subject by a user.

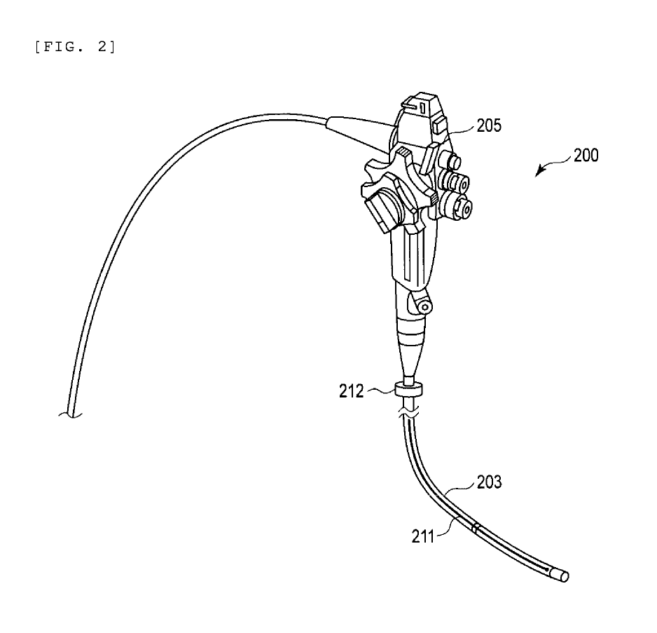

The endoscope 200 according to the embodiment is, for example, a colonoscope. As illustrated in FIGS. 2 to 4, the endoscope 200 includes an insertion portion 203 as a flexible insert having an elongated shape, and a manipulation unit 205 provided at one end of the insertion portion 203. In the following description, a side, on which the manipulation unit 205 of the insertion portion 203 is provided, is referred to as a rear end side, and the other end is referred to as a front end side.

The insertion portion 203 is provided with a camera on the front end side, and an image is acquired by the camera. The captured image is subjected to various types of common image processing, and then is displayed on the display device 320. The insertion portion 203 is provided with a bending portion on a front end portion thereof, and the bending portion bends in response to manipulation of the manipulation unit 205. A user grips, for example, the manipulation unit 205 in the left hand, and inserts the insertion portion 203 into a subject while sending out or pulling on the insertion portion 203 in the right hand. In such an endoscope 200, the insertion portion 203 is provided with a sensor 201 in order to obtain positions of portions of the insertion portion 203 and a shape of the insertion portion 203.

Various sensors can be used as the sensor 201. An example of a configuration of the sensor 201 is described with reference to FIGS. 2 to 4.

FIG. 2 is a diagram illustrating a first example of the configuration of the sensor 201. In the first example, the insertion portion 203 is provided with a shape sensor 211 and an inserting amount sensor 212. The shape sensor 211 is a sensor for obtaining the shape of the insertion portion 203. It is possible to obtain the shape of the insertion portion 203 from an output of the shape sensor 211. The inserting amount sensor 212 is a sensor for obtaining an inserting amount as an amount of insertion of the insertion portion 203 into a subject. It is possible to obtain a position of a predetermined spot of the insertion portion 203 on the rear end side, which is measured by the inserting amount sensor 212, from an output of the inserting amount sensor 212. It is possible to obtain positions of portions of the insertion portion 203, based on the position of the predetermined spots of the insertion portion 203 on the rear end side and the shape of the insertion portion 203 including the positions.

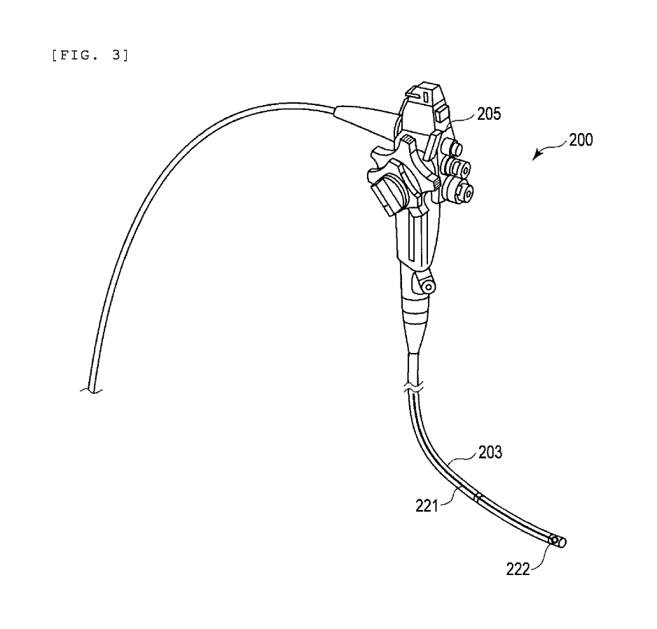

FIG. 3 is a diagram illustrating a second example of the configuration of the sensor 201. In the second example, the insertion portion 203 is provided with a shape sensor 221 and a position sensor 222 in order to obtain the shape of the insertion portion 203. The position sensor 222 detects a position of a spot in which the position sensor 222 is disposed. FIG. 3 illustrates an example in which the position sensor 222 is provided at the front end of the insertion portion 203. It is possible to calculate or estimate and obtain positions of the portions (arbitrary points), and the orientation and the bending shape of the insertion portion 203, based on the shape of the insertion portion 203, which is obtained based on an output of the shape sensor 221 and the position of the spot in which the position sensor 222, which is obtained based on the output from the position sensor 222, is provided.

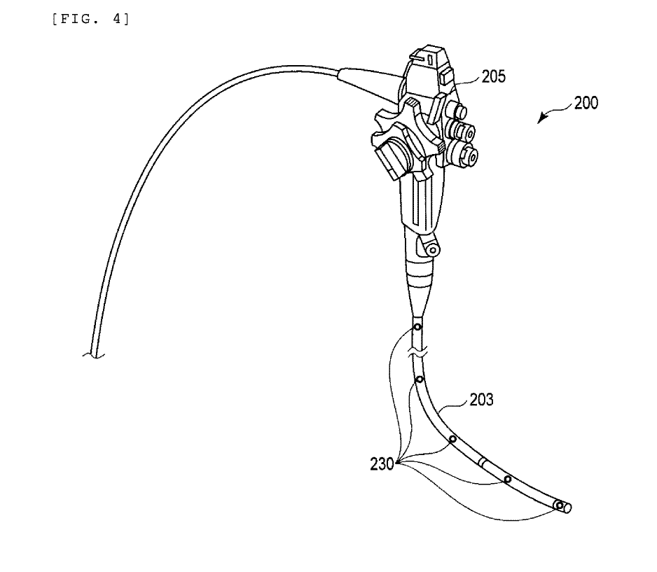

FIG. 4 is a diagram illustrating a third example of the configuration of the sensor 201. In the third example, the insertion portion 203 is provided with a plurality of position sensors 230 in order to obtain the positions of the portions of the insertion portion 203. It is possible to obtain the positions of predetermined spots of the insertion portion 203, in which the position sensor 230 is provided, from the outputs of the position sensors 230. It is possible to obtain the shape of the insertion portion 203 from a combination of the items of position information.

An example of a configuration of the shape sensors 211 and 221 are described with reference to FIG. 5. A shape sensor 260 provided in the insertion portion 203 according to the example includes a plurality of shape detection units 261. FIG. 5 illustrates an example employed in a case where four shape detection units 261 are provided, for simplicity. In other words, the shape sensor 260 includes a first shape detection unit 261-1, a second shape detection unit 261-2, a third shape detection unit 261-3, and a fourth shape detection unit 261-4. The number of shape detection units may not be limited to any number.

The shape detection unit 261 includes an optical fiber 262 provided along the insertion portion 203. The optical fiber 262 is provided with a reflective member 264 in an end portion on the front end side. The optical fiber 262 is provided with a branching portion 263 on the rear end side. The optical fiber 262 is provided with an incident lens 267 and a light source 265 at one branching end on the rear end side. The optical fiber 262 is provided with an emission lens 268 and a light detector 266 at the other branching end on the rear end side. In addition, the optical fiber 262 is provided with a detection region 269. The detection regions 269 includes a first detection region 269-1 provided in the first shape detection unit 261-1, a second detection region 269-2 provided in the second shape detection unit 261-2, a third detection region 269-3 provided in the third shape detection unit 261-3, and a fourth detection region 269-4 provided in the fourth shape detection unit 261-4, and the detection regions are disposed on different positions of the insertion portion 203 in a longitudinal direction thereof.

Light emitted from the light source 265 is incident to the optical fiber 262 via the incident lens 267. The light travels through the optical fiber 262 toward the front end direction and is reflected from the reflective member 264 provided on the front end. The reflected light travels through the optical fiber 262 toward the rear end side and is incident to the light detector 266 via the emission lens 268. The light propagation efficiency of the light in the detection region 269 changes depending on a bending state of the detection region 269. Therefore, it is possible to obtain the bending state of the detection region 269, based on a light quantity which is detected by the light detector 266.

It is possible to obtain a bending state of the first detection region 269-1, based on a light quantity which is detected by the light detector 266 of the first shape detection unit 261-1. Similarly, a bending state of the second detection region 269-2 is obtain, based on the light quantity which is detected by the light detector 266 of the second shape detection unit 261-2, a bending state of the third detection region 269-3 is obtained, based on the light quantity which is detected by the light detector 266 of the third shape detection unit 261-3, and a bending state of the fourth detection region 269-4 is obtained, based on a light quantity which is detected by the light detector 266 of the fourth shape detection unit 261-4. In this manner, it is possible to detect the bending states of the portions of the insertion portion 203, and it is possible to obtain the shape of the entire insertion portion 203.

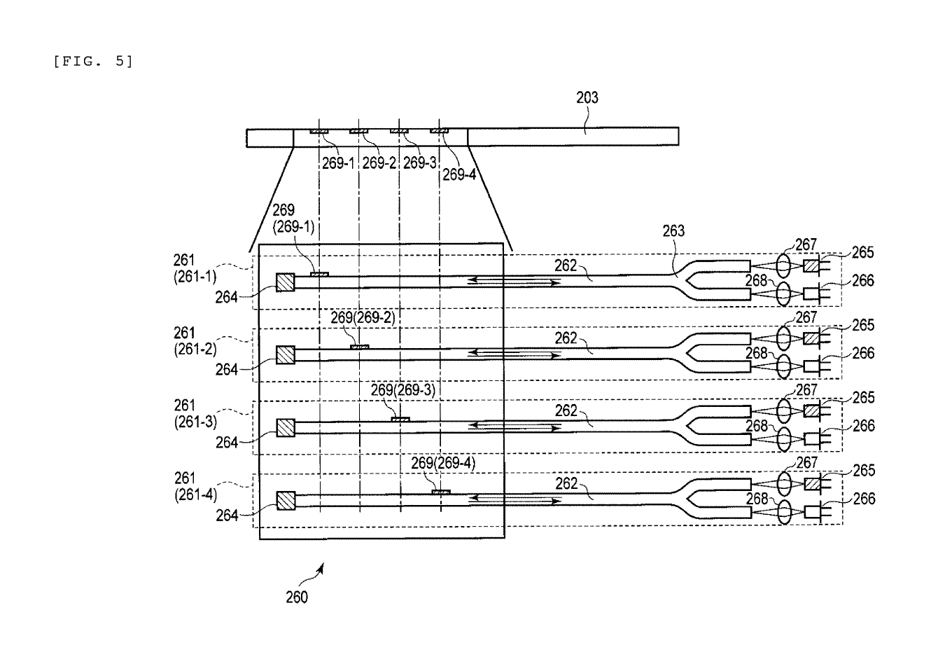

Next, an example of a configuration of the inserting amount sensor 212 is described with reference to FIGS. 6 and 7.

FIG. 6 is a diagram illustrating an example of the configuration of the inserting amount sensor 212. In the example, the inserting amount sensor 212 includes a holding member 241 that is fixed to an insertion opening of the subject. The holding member 241 is provided with a first encoder head 242 for detection in the inserting direction and a second encoder head 243 for detection in a torsion direction. An encoder pattern is formed in the insertion portion 203. The first encoder head 242 detects the inserting amount of the insertion portion 203 in the longitudinal direction during the insertion, based on the encoder pattern formed on the insertion portion 203. The second encoder head 243 detects a rotation amount of the insertion portion 203 in a circumferential direction during the insertion, based on the encoder pattern formed on the insertion portion 203.

FIG. 7 is a diagram illustrating another example of the configuration of the inserting amount sensor 212. In the example, the inserting amount sensor 212 includes a first roller 246 for detection in the inserting direction, a first encoder head 247 for detection in the inserting direction, a second roller 248 for detection in the torsion direction, a second encoder head 249 for detection in the torsion direction. The first roller 246 rotates in response to movement of the insertion portion 203 in the longitudinal direction. An encoder pattern is formed in the first roller 246. The first encoder head 247 is disposed to face the first roller 246. The first encoder head 247 detects the inserting amount of the insertion portion 203 in the longitudinal direction during the insertion, based on a rotation amount of the first roller 246 rotating in response to the insertion. The second roller 248 rotates in response to rotation of the insertion portion 203 in the circumferential direction. An encoder pattern is formed in the second roller 248. The second encoder head 249 is disposed to face the second roller 248. The second encoder head 249 detects the rotation amount of the insertion portion 203 in the circumferential direction during the insertion, based on the rotation amount of the second roller 248 rotating in response to the rotation.

With the inserting amount sensor 212 illustrated in FIGS. 6 and 7, a portion of the insertion portion 203 and a rotating angle of the portion can be identified at the position of the inserting amount sensor 212, with the position of the inserting amount sensor 212 as a reference. In other words, it is possible to identify a position of any portion of the insertion portion 203.

Next, the position sensors 222 and 230 are described. The position sensors 222 and 230 includes, for example, a coil which is provided in the insertion portion 203 and produces magnetism, and a reception device configured to be provided outside the subject. The reception device detects a magnetic field formed by the magnetic coil, and thereby it is possible to obtain a position of the coil. The position sensor is not limited to the sensor using the magnetism. The position sensor can have various configurations in which a wave transmitter, which is provided in the insertion portion 203 and transmits any of light waves, sound waves, electromagnetic waves, and the like, and a receiver, which is provided outside the subject and receives a signal transmitted from the wave transmitter, are included.

As described above, the following information is obtained, based on an output of the sensor 201 including a combination of the shape sensor, the inserting amount sensor, and the position sensor. The obtained information is described with reference to FIG. 8. It is possible to obtain, for example, a position of a front end 510 of the insertion portion 203 by using the sensor 201. The position of the front end 510 can be represented, for example, by a coordinate with the insertion opening in the subject as a reference.

For example, in the first example in which the shape sensor 211 and the inserting amount sensor 212 are provided as illustrated in FIG. 2, it is possible to obtain the position of the insertion portion 203 which is positioned in the insertion opening of the subject, based on the output of the inserting amount sensor 212. With the position as the reference, it is possible to obtain the position of the front end 510 of the insertion portion 203 with respect to the insertion opening of the subject, based on the shape of the insertion portion 203 which is obtained by the shape sensor 211.

For example, in the second example in which the shape sensor 221 and the position sensor 222 are provided as illustrated in FIG. 3, the position of the position sensor 222 in the insertion portion 203 is known. Therefore, it is possible to obtain the position of the front end 510 of the insertion portion 203 with respect to the position sensor 222 with the position as reference, further based on the shape of the insertion portion 203 which is obtained by the shape sensor 221. Since it is possible to obtain the position of the position sensor 222 with respect to the subject from the output of the position sensor 222, it is possible to obtain the position of the front end 510 of the insertion portion 203 with respect to the insertion opening of the subject. Note that, in a case where the position sensor 222 is provided at the front end 510 of the insertion portion 203, it is possible to directly obtain the position of the front end 510 of the insertion portion 203 with respect to the insertion opening of the subject, based on the output of the position sensor 222.

For example, in the third example in which the position sensor 230 is provided as illustrated in FIG. 4, it is possible to obtain the position of the front end 510 of the insertion portion 203 with respect to the insertion opening of the subject, based on the output of the position sensor 230 positioned in the vicinity of the front end of the insertion portion 203.

In addition, similar to the position of the front end 510 of the insertion portion 203, it is possible to obtain a position of an arbitrary spot 520 of the insertion portion 203 with respect to the insertion opening of the subject. In addition, in the description provided above, the reference position is the insertion opening of the subject; however, the reference position is not limited thereto. The reference position may be any position. A spot of the insertion portion 203, in which sensing is (directly) performed, is referred to as a “detection point”, and, in the embodiment, a spot of the insertion portion 203, in which information associated with a position is (directly) acquired, is referred to as the “detection point”.

In addition, it is possible to obtain the shape of the insertion portion 203, based on the output of the sensor 201. For example, as in the first example and the second example described above, in the case where the shape sensor 211 or 221 is provided, it is possible to obtain the shape of the insertion portion 203, based on the output of the sensor. In addition, as in the third example, in the case where the position sensors 230 are provided, the shape of the insertion portion 203 is obtained, based on information associated with the detected positions by the position sensors 230, at which the position sensors 230 are disposed, and results of calculation performed by interpolating the positions of the position sensors 230.

Further, when the shape of the insertion portion 203 is obtained, a position of a specific portion of the shape of the insertion portion 203 is obtained. For example, when a bending portion is defined as a region 530 having a predetermined shape, a position of a folding end 540 of the bending portion of the insertion portion 203 is obtained. Here, the folding end is determined as follows, for example. For example, as in an example illustrated in FIG. 8, the insertion portion 203 moves upward, then bends, and moves downward in the figure. The folding end can be defined, for example, as a point located at the highest position in FIG. 8. As described above, when the insertion portion 203 bends, the folding end can be defined as a point located at the farthest end in a predetermined direction. A point of the insertion portion 203, of which sensing information needs to be obtained in a direct or estimating manner, is referred to as an “attention point”. In the embodiment, attention is paid to a characteristic “attention point” that is determined, based on the shape of the insertion portion 203. The attention point is not limited to the folding end, and may be any point as a characteristic point which is determined, based on the shape of the insertion portion 203.

Since the information described above is acquired, based on the output of the sensor 201, the insertion-extraction support device 100 according to the embodiment includes a position acquiring unit 110 and a shape acquiring unit 120 as illustrated in FIG. 1. The position acquiring unit 110 performs processing on position information associated with the portions of the insertion portion 203. The position acquiring unit 110 includes a detection point acquiring unit 111. The detection point acquiring unit 111 identifies a position of the detection point. In addition, the position acquiring unit 110 is not limited to identifying the detection point, and can identify a position of the attention point as an arbitrary spot of the insertion portion 203, which is obtained from the output of the sensor 201 or the like. The shape acquiring unit 120 performs processing on information associated with the shape of the insertion portion 203. The shape acquiring unit 120 includes an attention point acquiring unit 121. The attention point acquiring unit 121 identifies the position of the attention point obtained based on the shape, based on the shape of the insertion portion 203 and the position information calculated by the position acquiring unit 110.

In addition, the insertion-extraction support device 100 includes a state determination unit 130. The state determination unit 130 calculates information associated with a state of the insertion portion 203 or a state of the subject into which the insertion portion 203 is inserted, using the information associated with the position of the detection point or the position of the attention point. To be more specific, as will be described below, whether or not the insertion portion 203 moves in compliance with the shape of the insertion portion 203, that is, whether or not the insertion portion has a self-compliance property, is evaluated by using various methods. The information associated with the state of the insertion portion 203 or the state of the subject, into which the insertion portion 203 is inserted, is calculated, based on the evaluation results.

The insertion-extraction support device 100 includes a support information generating unit 180.

The support information generating unit 180 generates information associated with support for the insertion of the insertion portion 203 into the subject by a user, based on the information associated with the state of the insertion portion 203 or the subject which is calculated by the state determination unit 130. The support information generated by the support information generating unit 180 is represented by characters or figures and is displayed on the display device 320. In addition, the support information generating unit 180 generates various types of information used for the control of the operation of the endoscope 200 by the control device 310, based on the information associated with the state of the insertion portion 203 or the subject which is calculated by the state determination unit 130.

The insertion-extraction support device 100 further includes a program memory 192 and a temporary memory 194. In the program memory 192, a program for an operation of the insertion-extraction support device 100, a predetermined parameter, or the like is recorded. The temporary memory 194 is used for temporary storage during the calculation of the units of the insertion-extraction support device 100.

The insertion-extraction support device 100 further includes a recording device 196. The recording device 196 records support information generated by the support information generating unit 180. The recording device 196 is not limited to being disposed in the insertion-extraction support device 100. The recording device 196 may be provided outside the insertion-extraction support device 100. The support information is recorded in the recording device 196, and thereby the following effects are achieved. In other words, it is possible to reproduce or analyze the information associated with the state of the insertion portion 203 or the subject afterward, based on the support information recorded in the recording device 196. In addition, the information recorded in the recording device 196 can be used as reference information or history information when the insertion is performed into the same subject.

For example, the position acquiring unit 110, the shape acquiring unit 120, the state determination unit 130, the support information generating unit 180, or the like includes a circuit such as a central processing unit (CPU), an application specific integrated circuit (ASIC), or the like.

Next, calculation of the information associated with the state of the insertion portion 203 or the subject will be described with reference to a specific example.

In a first state determination method, the state of the insertion portion 203 is determined, based on positional relationships between the detection points.

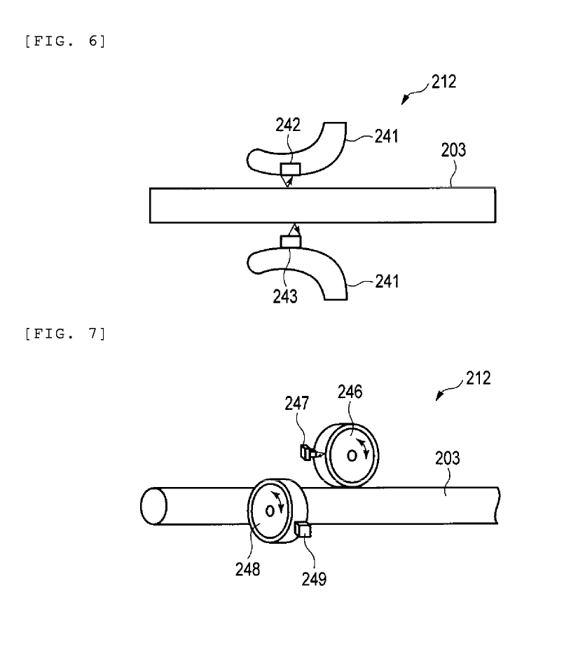

FIG. 9 schematically illustrates a state of movement of the insertion portion 203 between a time point t1 and a time point t2. A solid line represents the state of the insertion portion 203 at the time point t1, and a dashed line represents the state of the insertion portion 203 at the time point t2. In the example described here, positions of the front end portion and an arbitrary spot on the rear end side of the insertion portion 203 are identified as the attention point. The portion on the arbitrary spot on the rear end side, as a predetermined portion, is referred to as a rear-side attention point. Note the position, at which the position sensor is disposed, is set as the rear-side attention point. In other words, a case where the rear-side attention point is the detection point is described with reference to an example. Hereinafter, the point is referred to as a rear-side detection point. In addition, one attention point is not limited to being positioned in the front end portion, and may be an arbitrary spot on the front end side; however, here, the point is described as the front end. Note a case where the position sensor is disposed in the front end portion is described with reference to an example. In other words, a case where the front end portion is also the detection point is described with reference to an example.

At the time point t1, the front end portion of the insertion portion 203 is located at a first front end position 602-1. At the time point t1, the rear-side detection point of the insertion portion 203 is located at a first rear end position 604-1. At the time point t2 after a period of time Δt elapses from the time point t1, the front end portion of the insertion portion 203 is located at a second front end position 602-2. At the time point t2, the rear-side detection point of the insertion portion 203 is located at a second rear end position 604-2.

Here, a displacement from the first front end position 602-1 to the second front end position 602-2, that is, a displacement of the front end portion, is represented by ΔX21. A displacement from the first rear end position 604-1 to the second rear end position 604-2, that is, a displacement of the rear-side detection point, is represented by ΔX11. As illustrated in FIG. 9, when the insertion portion 203 is inserted into the subject, |ΔX21|≈|ΔX11|.

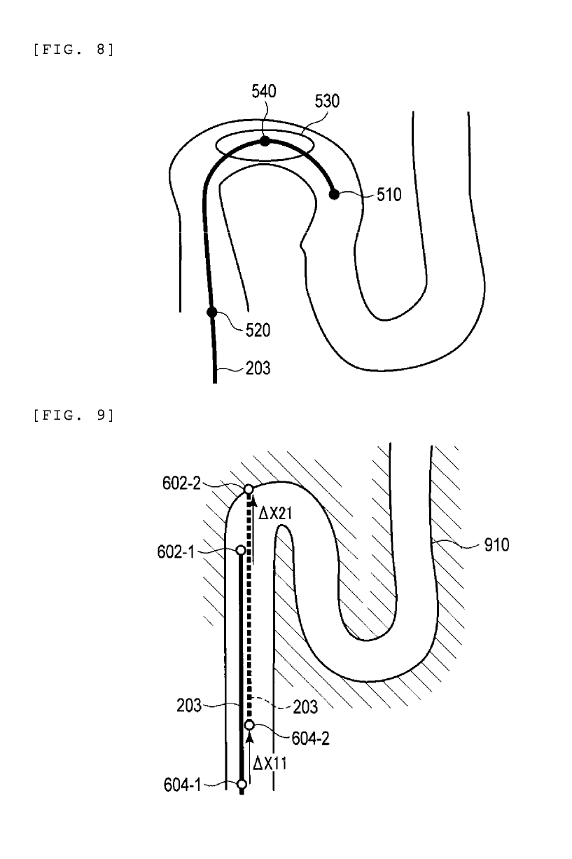

FIG. 10 is a schematic diagram illustrating a case where the insertion portion 203 is inserted into a subject 910 in a bending region 914 in which the subject bends. At a time point t3 after the period of time Δt elapses from the time point t2, the front end portion of the insertion portion 203 is located at a third front end position 602-3. At the time point t3, the rear-side detection point of the insertion portion 203 is located at a third rear end position 604-3. Here, a displacement from the second front end position 602-2 to the third front end position 602-3, that is, a displacement of the front end portion, is represented by ΔX22. A displacement from the second rear end position 604-2 to the third rear end position 604-3, that is, a displacement of the rear-side detection point, is represented by ΔX12. As illustrated in FIG. 10, when the insertion portion 203 is inserted along the subject, |ΔX22|≈|ΔX12|.

FIG. 11 is a schematic diagram illustrating a case where the insertion portion 203 is not inserted along the subject in the bending region 914 in which the subject bends. At the time point t3 after the period of time Δt elapses from the time point t2, the front end portion of the insertion portion 203 is located at a third front end position 602-3′. At the time point t3, the rear-side detection point of the insertion portion 203 is located at a third rear end position 604-3′. Here, a displacement from the second front end position 602-2 to the third front end position 602-3′, that is, a displacement of the front end portion, is represented by ΔX22′. A displacement from the second rear end position 604-2 to the third rear end position 604-3′, that is, a displacement of the rear-side detection point, is represented by ΔX12′. As illustrated in FIG. 11, when the insertion portion 203 is not inserted along the subject, |ΔX22′|≈|ΔX12′| (|ΔX22′|<|ΔX12′|).

Note that, in FIGS. 9 to 11, a time change from the time point t1 to the time point t2 and a time change from the time point t2 to the time point t3 are the same value Δt in the example such that the calculation is performed in automatic measurement; however, the value may not necessarily be the same value. The same is true of the following examples.

In the case illustrated in FIG. 11, the front end of the insertion portion 203 is pressed or compressed by the subject 910 as illustrated by an outline arrow in FIG. 11. Conversely, in the front end portion of the insertion portion 203, the insertion portion 203 is significantly pressed against the subject 910. In addition, in the case illustrated in FIG. 11, a region 609 between the front end portion of the insertion portion 203 and the rear-side detection point buckles.

When a moving amount of the rear-side detection point as the detection point of the insertion portion 203 on the rear end side is equal to a moving amount of the front end portion as the detection point on the front end side, that is, when there is a high interlocking condition between the moving amount of the rear-side detection point and the moving amount of the front end portion, the insertion portion 203 turns out to be smoothly inserted along the subject 910. On the other hand, when the moving amount of the front end portion is smaller than the moving amount of the rear-side detection point, that is, when there is a low interlocking condition between the moving amount of the rear-side detection point and the moving amount of the front end portion, the front end portion of the insertion portion 203 turns out to be stuck. In addition, it turns out that there is a possibility that an unintended abnormality occurs between the two detection points, that is, between the front end portion and the rear-side detection point. As described above, the buckling of the insertion portion 203, a size of a pressing force of the insertion portion against the subject, or the like is clearly known, based on analysis of positional relationships between the detection points using the first state determination method. In other words, it is possible to acquire the information associated with the state of the insertion portion or the subject using the first state determination method.

First manipulation support information α1 is introduced as a value representing the state of the insertion portion 203 as described above. For example, when the displacement of the front end portion is ΔX2, and the displacement of the rear-side detection point is ΔX1, the first manipulation support information α1 can be defined as follows.

α1≡|ΔX2|/|ΔX1|

The first manipulation support information α1 indicates that the insertion portion 203 is inserted into the subject 910, as the value approximates to 1.

In addition, the first manipulation support information α1 may be defined as follows.

α1≡(|ΔX2|+C2)^{L}/(|ΔX1|+C1)^{M}

Here, C1, C2, L, and M are arbitrary real numbers, respectively.

For example, in a case where detected noise component levels of ΔX1 and ΔX2 are N1 and N2 (N1 and N2≧0), parameter C1·C2·L·M is set as follows.

C1=N1^{·}|ΔX1|≧N1

C2=−N2|ΔX2|≧N2

=−|ΔX2||ΔX2|<N2

L=M=1

For example, N1 or N2 may be set to a value of about three times a standard deviation (σ) of the noise level.

Setting in which C1 is positive and C2 is negative is performed against noise as described above, thereby reducing an effect of the detection noise, and the first manipulation support information α1, with which false detection due to the detection noise is lowered, is obtained. In addition, a method of reducing the noise effect can also be applied to a case of other support information calculations which will be described below.

Note that, in a case where the endoscope 200 is the colonoscope, and thus the subject 910 is the large intestine, the bending region 914 described above corresponds to the uppermost portion (so-called “S-top”) of the S-shaped colon, for example.

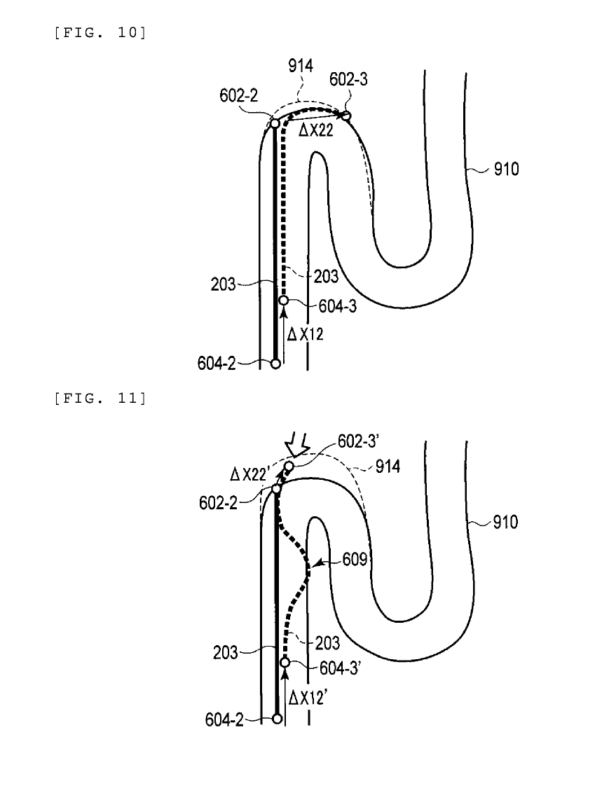

FIG. 12 schematically illustrates an example of a configuration of the insertion-extraction support device 100 for executing the first state determination method.

The insertion-extraction support device 100 includes the position acquiring unit 110 that has the detection point acquiring unit 111, the state determination unit 130, and the support information generating unit 180. The detection point acquiring unit 111 obtains positions of the detection points, based on the information output from the sensor 201.

The state determination unit 130 includes a displacement information acquiring unit 141, an interlocking condition calculation unit 142, and a buckling determination unit 143. The displacement information acquiring unit 141 calculates displacements of the detection points, based on the positions of the detection points which are obtained as time elapses. The interlocking condition calculation unit 142 calculates the displacements of the detection points and interlocking conditions between the detection points, based on interlocking condition information 192-1 recorded in the program memory 192. The interlocking condition information 192-1 has, for example, a relationship between differences of the displacements of the detection points and an evaluation value of the interlocking condition. The buckling determination unit 143 determines a buckling state of the insertion portion 203, based on the calculated interlocking condition, and determination reference information 192-2 recorded in the program memory 192. The determination reference information 192-2 has, for example, a relationship between the interlocking conditions and the buckling state.

The support information generating unit 180 generates the manipulation support information, based on the determined buckling state. The manipulation support information is subjected to feedback in control by the control device 310, is displayed on the display device 320, or is recorded in the recording device 196.

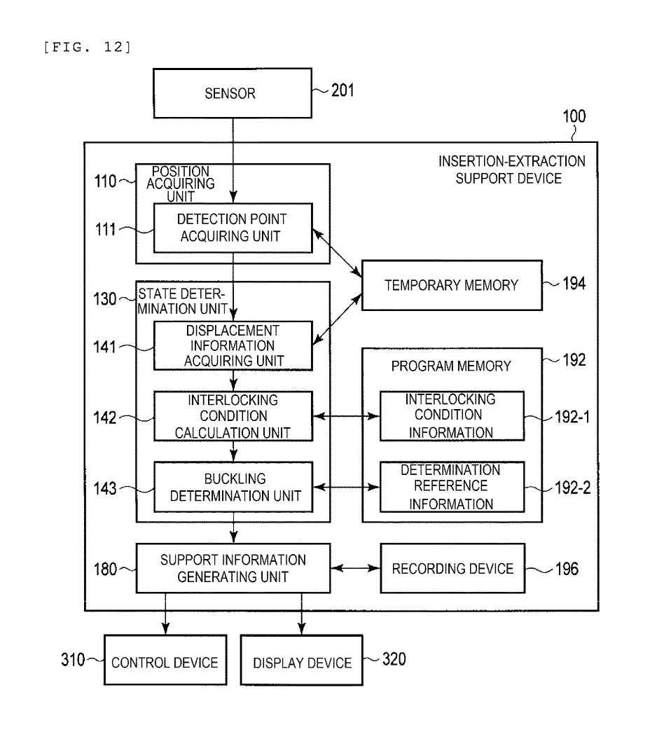

The operation of the insertion-extraction support device 100 in the first state determination method is described with reference to a flowchart illustrated in FIG. 13.

In Step S101, the insertion-extraction support device 100 acquires output data from the sensor 201. In Step S102, the insertion-extraction support device 100 obtains the positions of the detection points, based on the data acquired in Step S101.

In Step S103, the insertion-extraction support device 100 acquires successive changes in the positions of the detection points, respectively. In Step S104, the insertion-extraction support device 100 evaluates, for each detection point, a difference in the change in the position of the detection point. In other words, the interlocking condition in the positional change of the detection points is calculated. In Step S105, the insertion-extraction support device 100 evaluates the bucking regarding whether or not the buckling occurs between the detection point and the detection point, what a degree the buckling occurs, or the like, based on the interlocking condition calculated in Step S104.

In Step S106, the insertion-extraction support device 100 generates appropriate support information that is used in the following processes, based on the evaluation results of whether or not the buckling occurs or the like, and outputs the support information, for example, to the control device 310 or to the display device 320.

In step S107, the insertion-extraction support device 100 determines whether or not an end signal for ending the processes has been input. When the end signal is not input, the process returns to Step S101. In other words, the processes described above are repeated until the end signal is input and the manipulation support information is output. On the other hand, when the end signal is input, the corresponding process is ended.

The first state determination method is used, thereby positions of the two or more detection points are identified, and the manipulation support information indicating whether or not the abnormality occurs, such as whether the buckling of the insertion portion 203 occurs, can be generated, based on the interlocking conditions of the moving amounts.

In the example described above, the case where the manipulation support information is generated, based on the detection points, that is, the positions at which the sensing is directly performed, is described as an example. However, the configuration is not limited thereto. Searching support information may be generated using information associated with the attention point, that is, an arbitrary position of the insertion portion 203. In a case where the position of the attention point is used, the detection point acquiring unit 111 does not obtain the positions, but the position acquiring unit 110 obtains the positions of the attention points, and the obtained positions of the attention points are used. The other processes are the same.

In the example described above, the case of having two detection points is described. However, the number of detection points is not limited thereto, and may be any number. When the number of detection points increases, it is possible to acquire more detailed information associated with the state of the insertion portion 203. For example, as illustrated in FIG. 14, a case of having four detection points is described as follows. In other words, in the example, as illustrated in FIG. 14, the insertion portion 203 is provided with four detection points 605-1, 606-1, 607-1, and 608-1. When the insertion portion 203 is inserted along the subject 910 from the time point t1 to the time point t2, moving amounts ΔX51, ΔX61, ΔX71, and ΔX81 from the four detection points 605-1, 606-1, 607-1, and 608-1, respectively, at the time point t1 to four detection points 605-2, 606-2, 607-2, and 608-2, respectively, at the time point t2 are substantially equal to each other.

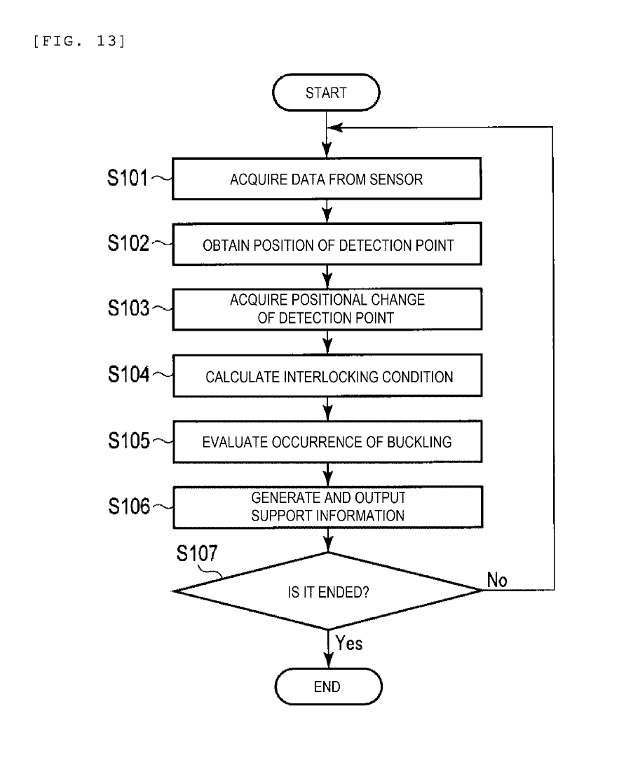

As illustrated in FIG. 15, when the insertion portion 203 is inserted along the subject 910 from the time point t2 to the time point t3, moving amounts ΔX52, ΔX62, ΔX72, and ΔX82 from the four detection points 605-2, 606-2, 607-2, and 608-2, respectively, at the time point t2 to four detection points 605-3, 606-3, 607-3, and 608-3, respectively, at the time point t3 are substantially equal to each other.

Meanwhile, as illustrated in FIG. 16, when the insertion portion 203 is inserted along the subject 910 from the time point t2 to the time point t3, moving amounts ΔX52′, ΔX62′, ΔX72′, and ΔX82′ from the four detection points 605-2, 606-2, 607-2, and 608-2, respectively, at the time point t2 to four detection points 605-3′, 606-3′, 607-3′, and 608-3′, respectively, at the time point t3 are not substantially equal to each other. In other words, a first moving amount Δ52′ of the detection point 605 on the forefront end side, a second moving amount Δ62′ of the second detection point 606 from the front end, a third moving amount Δ72′ of the third detection point 607 from the front end, and a fourth moving amount Δ82′ of the detection point 608 on the rearmost end side are different from each other. Further, the first moving amount Δ52′ and the second moving amount Δ62′ are substantially equal to each other, the third moving amount Δ72′ and the fourth moving amount Δ82′ are substantially equal to each other, the second moving amount Δ62′ and the third moving amount Δ72′ are significantly different from each other, and equal to each other, |Δ62′|<|Δ72′|. From the results, an occurrence of the buckling between the second detection point 606 from the front end and the third detection point 607 from the front end is determined. As described above, when the number of detection points increases, an amount of information increases, and more detailed information associated with the state of the insertion portion 203 is acquired. When the number of detection points increases, the spot of insertion portion 203, at which the buckling occurs, can be identified.

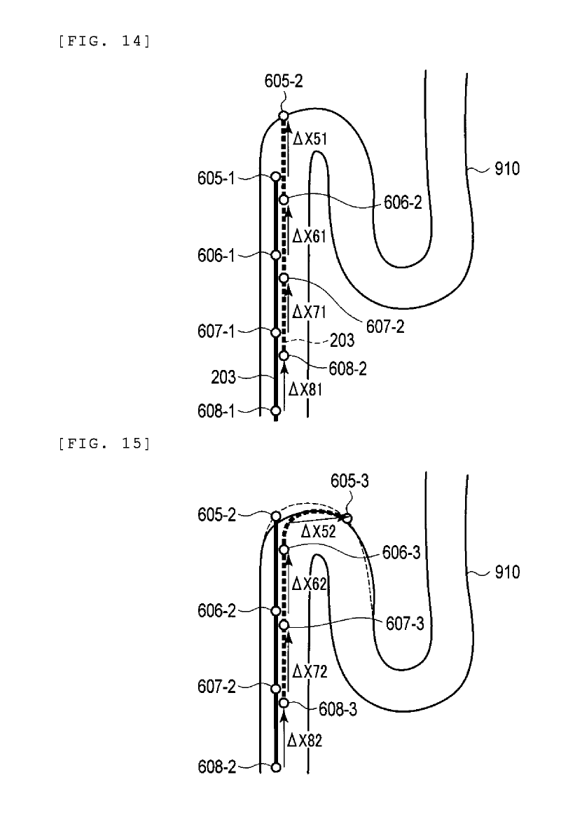

Regardless of insertion of the rear end side of the insertion portion 203, a case where the front end portion is stuck is not limited to the case where the insertion portion 203 buckles in the subject, and, for example, as illustrated in FIG. 17, the insertion portion 203 also causes a bending region of the subject to be also deformed (extended). Here, FIG. 17 schematically illustrates the shape of the insertion portion 203 at a time point t4 and the shape of the insertion portion 203 at a time point t5 after the period of time Δt elapses from the time point t4. Even in this case, a second moving amount ΔX23 as a difference between the position 602-4 in the front end portion at the time point t4 and the position 602-5 in the front end portion at the time point t5 is smaller than a first moving amount ΔX13 as a difference between the position 604-4 on the rear end side at the time point t4 and the position 604-5 on the rear end side at the time point t5.

In other words, the interlocking conditions between the moving amounts of the two detection points are lowered.

As described above, according to the first state determination method, the detection is not limited to the buckling, and it is possible to detect a change in an insertion state which is not an intended detection target, such as the deformation of the subject 910 by the insertion portion 203.

In a second state determination method, the state of the insertion portion 203 is determined, based on a displacement of a characteristic attention point which is identified due to the shape.

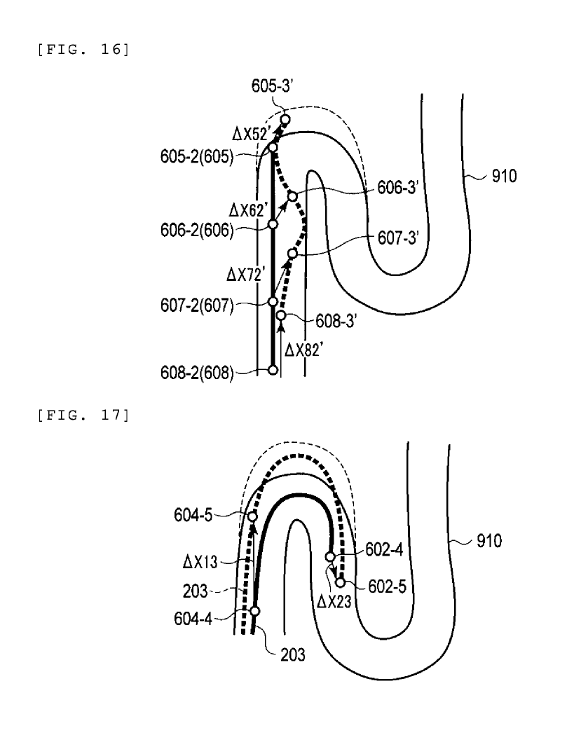

FIG. 18 schematically illustrates the shape of the insertion portion 203 at the time point t1 and the shape of the insertion portion 203 at the time point t2 after the period of time Δt elapses from the time point t1. At this time, an arbitrary spot of the insertion portion 203 on the rear end side moves from a first rear end position 614-1 to a second rear end position 614-2. In the following description, the arbitrary spot on the rear end side is described as a position of the position sensor disposed on the rear end side. The position is referred to as the rear-side detection point. Meanwhile, the front end of the insertion portion 203 moves from a first front end position 612-1 to a second front end position 612-2.

FIG. 19 schematically illustrates the shape of the insertion portion 203 at the time point t2 and the shape of the insertion portion 203 at the time point t3 after the period of time Δt elapses from the time point t2. In the case illustrated in FIG. 19, the insertion portion 203 is inserted along the subject 910. In other words, the rear-side detection point of the insertion portion 203 moves by a distance ΔX1 from a second rear end position 614-2 to a third rear end position 614-3. At this time, the front end of the insertion portion 203 moves by a distance ΔX2 along the insertion portion 203 from the second front end position 612-2 to the third front end position 612-3.

Here, the folding end (position illustrated uppermost side in FIG. 19) of a bending region of the insertion portion 203 is set as an attention point 616. At this time, first, the shape of the insertion portion 203 is identified and the position of the attention point 616 is identified, based on the identified shape.

In the case illustrated in FIG. 19, the position of the attention point 616 does not change even when the position of the rear-side detection point of the insertion portion 203 changes. In other words, between the time point t2 and the time point t3, the insertion portion 203 is inserted along the subject 910, and the insertion portion 203 is inserted so as to slide in the longitudinal direction thereof. Hence, between the time point t2 and the time point t3, the position of the attention point 616 does not change.

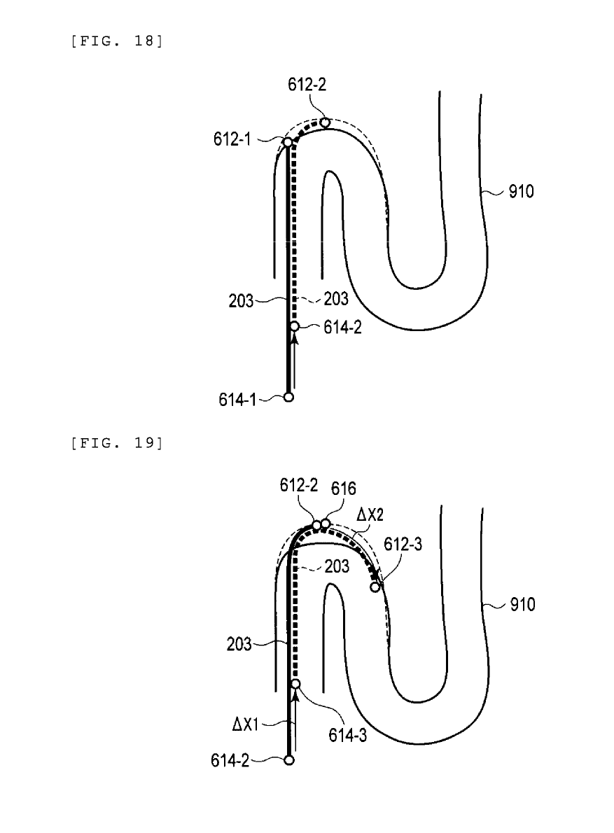

FIG. 20 schematically illustrates another example of the shape of the insertion portion 203 at the time point t2 and the shape of the insertion portion 203 at the time point t3 after the period of time Δt elapses from the time point t2. In the case illustrated in FIG. 20, the insertion portion 203 is not inserted along the subject 910. In other words, the rear-side detection point of the insertion portion 203 moves by a distance ΔX3 from the second rear end position 614-2 to a third rear end position 614-3′. At this time, the front end of the insertion portion 203 moves upward in FIG. 20 by a distance ΔX5 from the second front end position 612-2 to the third front end position 612-3′.

The state illustrated in FIG. 20 can occur, for example, in a case where the front end portion of the insertion portion 203 is caught in the subject 910, and thus the insertion portion 203 does not move forward in the longitudinal direction thereof. At this time, the subject 910 is pushed in response to the insertion of the insertion portion 203. As a result, the position of the attention point 616 displacements by a distance ΔX4 toward the folding end side of the insertion portion 203 from the first position 616-1 to the second position 616-2 in response to the displacement of the position of the rear-side detection point of the insertion portion 203. In other words, the subject 910 is extended.

In the state illustrated in FIG. 20, the shape of the insertion portion 203 remains as a “stick shape”, and the subject 910 is pushed up in a region of a “grip” of the “stick”. This state is referred as the stick state.

As clearly understood from a comparison between the case illustrated in FIG. 19 and the case illustrated in FIG. 20, whether the insertion portion 203 is inserted along the subject or is not inserted along the subject can be determined, based on the change in the position of the attention point. In the example described above, a case where the insertion portion 203 performs parallel movement in the stick state is described; however, when the insertion portion 203 is deformed, the moving amount of the rear-side detection point is different from the moving amount of the attention point. In addition, an extending state of the subject 910 can be determined, based on the change in the position of the attention point. In addition, the time when the subject is extended means the time when the insertion portion 203 presses or compresses the subject 910. In other words, as illustrated by an outline arrow in FIG. 20, the subject 910 presses the insertion portion 203. Conversely, the insertion portion 203 presses the subject 910. Hence, a magnitude of pressure applied on the subject is clearly known, based on the change in the position of the attention point.

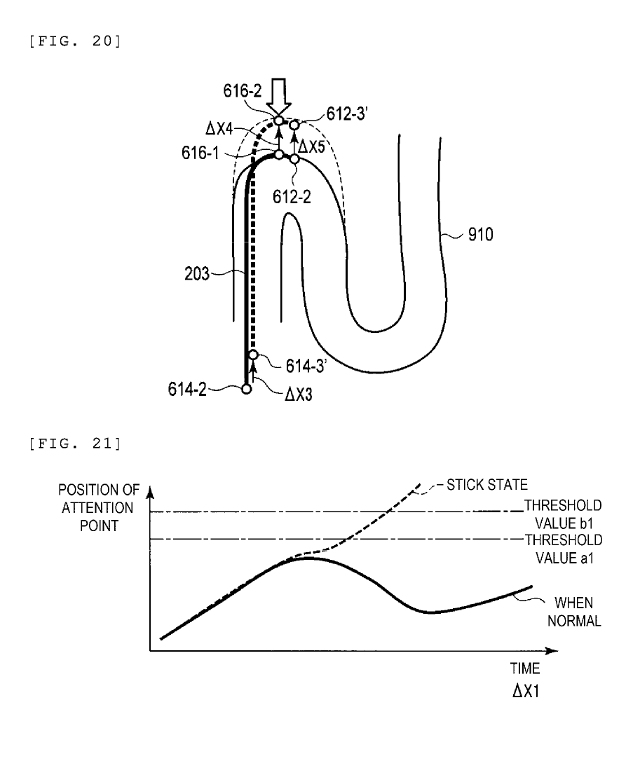

FIG. 21 illustrates the change in the position of the attention point as time elapses or with respect to a moving amount ΔX1 of the detection point. FIG. 21 illustrates the position of the attention point, for example, with the folding end direction as a plus direction. When the insertion portion 203 is normally inserted as represented by a solid line, the position of the attention point changes to have a value lower than a threshold value a1. By comparison, in the stick state represented by a dashed line, the position of the attention point changes to exceed the threshold value a1.

Regarding the value of the position of the attention point, it is possible to appropriately set threshold values, such as the threshold value al that is set as a value indicating that a warning that the subject 910 starts to be extended needs to be output, and a threshold value b1 that is set as a value indicating that a warning that there is a danger to the subject, if the subject 910 is further extended, needs to be output. Appropriate setting of the threshold value enables the information associated with the position of the attention point to be used as information for supporting the manipulation of the endoscope 200, such as an output of a warning to a user or a warning signal to the control device 310.

Second manipulation support information α2 is introduced as a value representing the state of the insertion portion 203 as described above. For example, when the displacement of the attention point is ΔXc, and the displacement of the rear-side detection point is ΔXd, the second manipulation support information α2 can be defined as follows.

α2≡|ΔXc|/|ΔXd|

The second manipulation support information α2 indicates that the insertion portion 203 is inserted along the subject 910, as the value approximates to 0, and indicates that the insertion portion 203 presses the subject 910 as the value approximates to 1.

In addition, the second manipulation support information α2 may be defined as follows.

α2≡(ΔXc+C2)^{L}/(|ΔXd|+C1)^{M}

Here, C1, C2, L, and M are arbitrary real numbers, respectively.

For example, a case where detected noise component levels of ΔXd and ΔXc are Nd and Nc (Nd and Nc≧0), a pushing amount with which no load is applied from a state in which the insertion portion comes into comes into contact with the subject is represented by P, and Nd<k1·P (here, 1≧k2>>k1≧0) using a parameter k1·k2.

When |ΔXd|<k2·P at any timing, movement amounts for predetermined periods of time to the timing or the predetermined number of times are accumulated and ΔXd and ΔXc are calculated such that |ΔXd|≧k2·P. At this time (that is, when |ΔXd|≧k2·P), the parameter C1·C2·L·M is set as follows.

C1=−Nd

C2=Nc

L=M=2

For example, N1 or N2 may be set to a value of about three times a standard deviation (σ) of the noise level.

Such setting is performed, and thereby the second manipulation support information α2, in which an effect of undetected movement is reduced with respect to a certain amount of movement, based on the detection noise, is obtained.

Further, measurement is performed such that k2·P<<|ΔXd|<P, and thereby it is possible to obtain the second manipulation support information α2 in a range in which no or a small load is applied to the subject. In addition, a method of reducing the noise effect can also be applied to a case of other support information calculations.

FIG. 22 schematically illustrates an example of a configuration of the manipulation support device for executing the second state determination method.

The insertion-extraction support device 100 includes the position acquiring unit 110, the shape acquiring unit 120, the state determination unit 130, and the support information generating unit 180. The detection point acquiring unit 111 of the position acquiring unit 110 obtains, for example, the position of the detection point as the spot of the insertion portion 203 on the rear end side, at which the position sensor is disposed, based on the information output from the sensor 201. The shape acquiring unit 120 obtains the shape of the insertion portion 203, based on the information output from the sensor 201. The attention point acquiring unit 121 of the shape acquiring unit 120 obtains the position of the attention point which is the folding end in the bending region of the insertion portion 203, based on the shape of the insertion portion 203.

The state determination unit 130 includes a displacement acquiring unit 151, a displacement information calculation unit 152, and an attention point state determination unit 153. The displacement acquiring unit 151 calculates the displacement of the attention point, based on the positions of the attention point obtained as time elapses, and displacement analysis information 192-3 recorded in the program memory 192. In addition, the displacement acquiring unit 151 calculates the displacement of the detection point, based on the positions of the detection point obtained as time elapses, and the displacement analysis information 192-3 recorded in the program memory 192. As described above, the displacement acquiring unit 151 functions as a first displacement acquiring unit that obtains a first displacement of the attention point, and further functions as a second displacement acquiring unit that obtains a second displacement of the detection point.

The displacement information calculation unit 152 calculates displacement information, based on the calculated displacement of the attention point and the calculated displacement of the detection point. The attention point state determination unit 153 calculates a state of the attention point, based on the calculated displacement information and support information determining reference information 192-4 recorded in the program memory 192.

The support information generating unit 180 generates the manipulation support information, based on the determined state of the attention point. The manipulation support information is subjected to feedback in control by the control device 310, is displayed on the display device 320, or is recorded in the recording device 196.

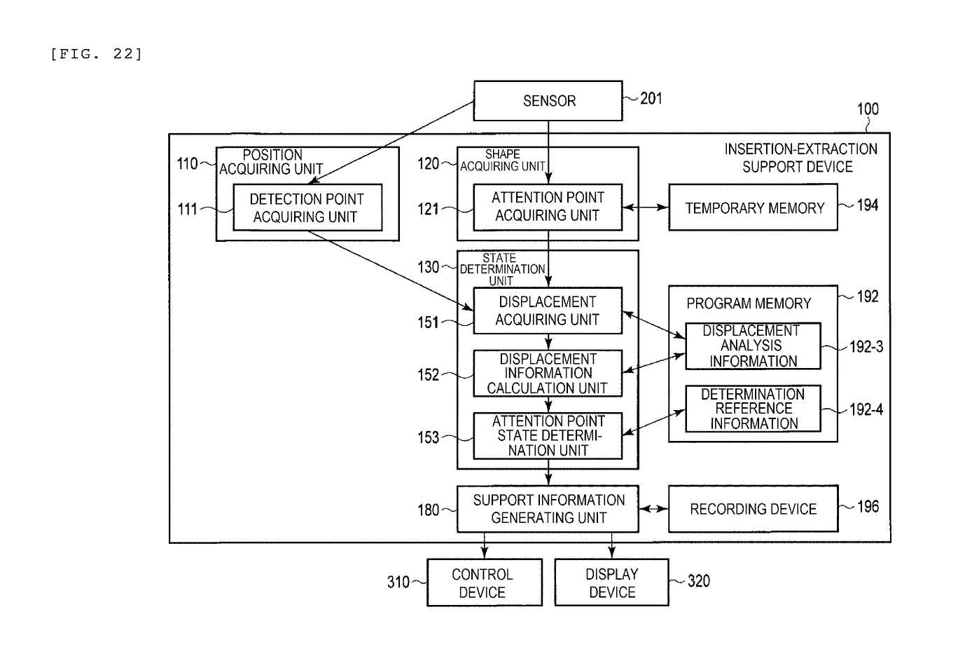

The operation of the insertion-extraction support device 100 in the second state determination method is described with reference to a flowchart illustrated in FIG. 23.

In Step S201, the insertion-extraction support device 100 acquires the output data from the sensor 201. In Step S202, the insertion-extraction support device 100 obtains the position of the detection point on the rear end side, based on the data acquired in Step S201.

In Step S203, the insertion-extraction support device 100 obtains the shape of the insertion portion 203, based on the data acquired in Step S201. In Step S204, the insertion-extraction support device 100 obtains the position of the attention point, based on the shape of the insertion portion 203 obtained in Step S203.

In Step S205, the insertion-extraction support device 100 acquires successive changes in the position of the attention point. In Step S206, the insertion-extraction support device 100 calculates an evaluation value of the positional change in the attention point with respect to the second manipulation support information α2 or the like, based on the positional change in the detection point and the positional change in the attention point. In Step S207, the insertion-extraction support device 100 evaluates the extension such as whether or not the extension of the subject occurs or what a degree the extension occurs on the periphery of the attention point, based on the evaluation value calculated in Step S206.

In Step S208, the insertion-extraction support device 100 generates appropriate support information that is used in the following processes, based on the determination results of whether or not the extension of the subject occurs, the second manipulation support information α2, or the like, and outputs the support information, for example, to the control device 310 or to the display device 320.

In step S209, the insertion-extraction support device 100 determines whether or not an end signal for ending the processes has been input. When the end signal is not input, the process returns to Step S201. In other words, the processes described above are repeated until the end signal is input and the manipulation support information is output. On the other hand, when the end signal is input, the corresponding process is ended.

The second state determination method is used, thereby the displacement of the attention point is identified, and the manipulation support information indicating whether or not the extension occurs in the subject can be generated, based on the displacement. Note that, in the example described above, the case where the manipulation support information is generated, based on the detection point on the rear end side, that is, the positions at which the sensing is directly performed, is described as an example. However, the configuration is not limited thereto. Searching support information may be generated using information associated with the attention point, that is, an arbitrary position of the insertion portion 203. In a case where the position of the attention point is used, the detection point acquiring unit 111 does not obtain the positions, but the position acquiring unit 110 obtains the positions of the attention points, and the obtained positions of the attention points are used. The other processes are the same.

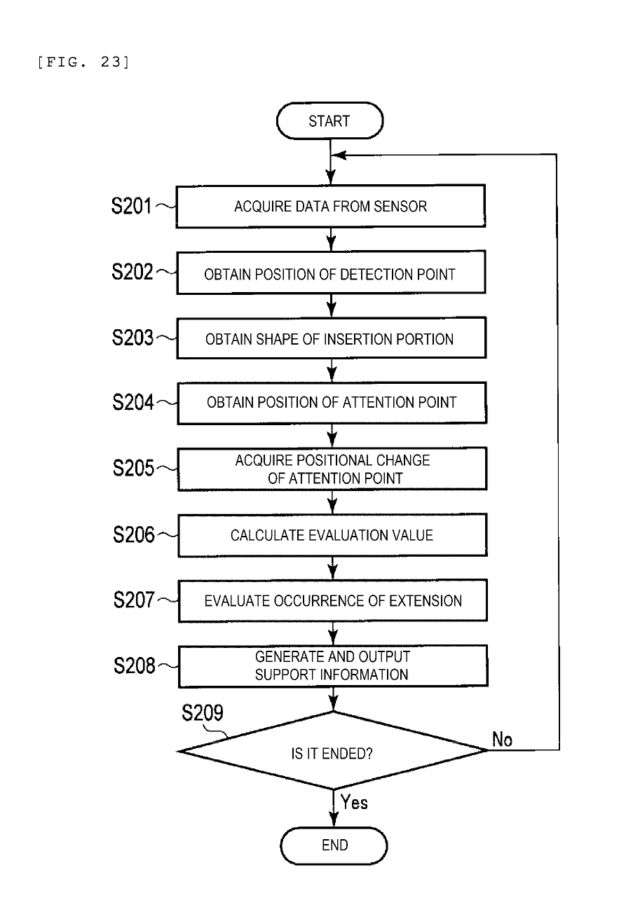

The attention point may be any spot of the insertion portion 203. Any position may be used as the attention point as long as characteristics in the shape of the insertion portion 203 is recognized such that the spot can be identified as the attention point. For example, as illustrated in FIG. 24, analysis may be performed on, in addition to a first attention point 617 identified in a bending region which is first formed when the insertion portion 203 is inserted into the subject 910, a second attention point 618 identified in a bending region which is formed when the insertion portion 203 is inserted into the subject. For example, as illustrated in FIG. 25, the position of the first attention point 617 does not change in response to the insertion of the insertion portion 203, but the position of the second attention point 618 changes in some cases. According to the second state determination method, in this case, a determination result that the extension does not occur at the first attention point 617, but the extension occurs at the second attention point 618 is output as the manipulation support information, based on the moving amount ΔX1 of the rear-side detection point and the moving amount ΔX2 of the second attention point 618.

Note that the attention point may be any position which is determined, based on the shape of the insertion portion 203. For example, the attention point may be the folding end of the bending region as in the example described above, may be a bending start position of the bending region, may be any position in a straight line-shaped region, for example, as an intermediate point between the bending region and the front end of the insertion portion 203, or may be an intermediate point or the like between a bending region and another bending region in a case where two or more bending regions occur. In any case, similar to the example described above, it is possible to output the manipulation support information. In addition, as the detection point, an arbitrary spot of the insertion portion 203 on the rear end side is described as an example thereof; however, the detection point is not limited thereto. The position of the detection point may be any position of the insertion portion 203.

In a third state determination method, the state of the insertion portion 203 is determined, based on a change in a position of the attention point on the insertion portion 203.

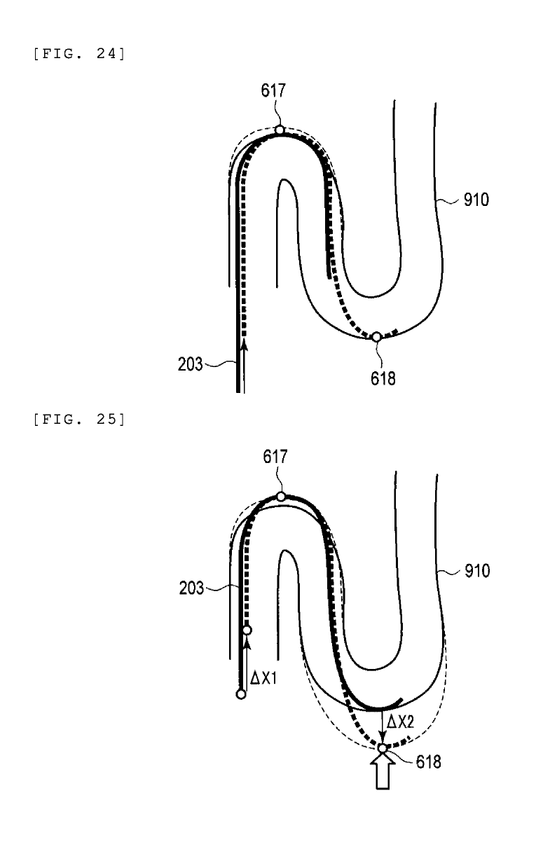

FIG. 26 schematically illustrates the shape of the insertion portion 203 at the time point t1 and the shape of the insertion portion 203 at the time point t2 after the period of time Δt elapses from the time point t1. At this time, an arbitrary spot of the insertion portion 203 on the rear end side moves by the distance ΔX1 from a first rear end position 624-1 to a second rear end position 624-2. A position, at which of the position sensor is disposed, will be described below as an example of the arbitrary spot on the rear end side. Hereinafter, the spot is referred to as the rear-side detection point. Meanwhile, the front end of the insertion portion 203 moves by the distance ΔX2 from a first front end position 622-1 to a second front end position 622-2. Ideally, the distance ΔX1 is equal to the distance ΔX2. The folding end of the region in which the insertion portion 203 bends at the time point t2 is set as an attention point 626-2. At this time, a point coincident with the attention point 626-2 in the insertion portion 203 is set as a second point 628-2. Here, the second point 628-2 can be described, for example, by a distance from the front end of the insertion portion 203, which is determined along a longitudinal axis of the insertion portion 203.

FIG. 27 schematically illustrates the shape of the insertion portion 203 at the time point t2 and the shape of the insertion portion 203 at the time point t3 after the period of time Δt elapses from the time point t2. In the case illustrated in FIG. 27, the insertion portion 203 is inserted along the subject 910. In this case, the rear-side detection point of the insertion portion 203 is inserted by the distance ΔX1.

The folding end of the region in which the insertion portion 203 bends at the time point t3 is set as an attention point 626-3. At this time, a point, which is a point on the insertion portion 203, is interlocked with the insertion and extraction of the insertion portion 203 so as to move together with the insertion portion, has a distance from the front end of the insertion portion 203, which does not change, and is coincident with the attention point 626-3, is set as a third point 628-3. Similar to the second point 628-2, the third point 628-3 can be described, for example, by the distance from the front end of the insertion portion 203.

In the example illustrated in FIG. 27, between the time point t2 and the time point t3, the point on the insertion portion 203 which represents the position of the attention point 626 moves by ΔSc in a rearward direction along the insertion portion 203, when viewed at a relative position from the front end of the insertion portion 203 from the second point 628-2 to the third point 628-3. When the insertion portion 203 is completely inserted along the subject, a displacement ΔSc from the second point 628-2 to the third point 628-3, which both represent the positions of the attention point 626 in the insertion portion 203, becomes equal to the displacement ΔX1 of the rear-side detection point of the insertion portion 203. A state in which the insertion portion 203 is inserted along the subject is referred to as a state in which the self-compliance property is maintained.

Even when the insertion portion 203 is not completely inserted along the subject, a displacement ΔSc from the second point 628-2 to the third point 628-3 becomes substantially equal to the displacement ΔX1 of the rear-side detection point of the insertion portion 203 when the insertion portion 203 is inserted substantially along the subject as illustrated in FIG. 27. In such a state, the self-compliance property is known to be high.

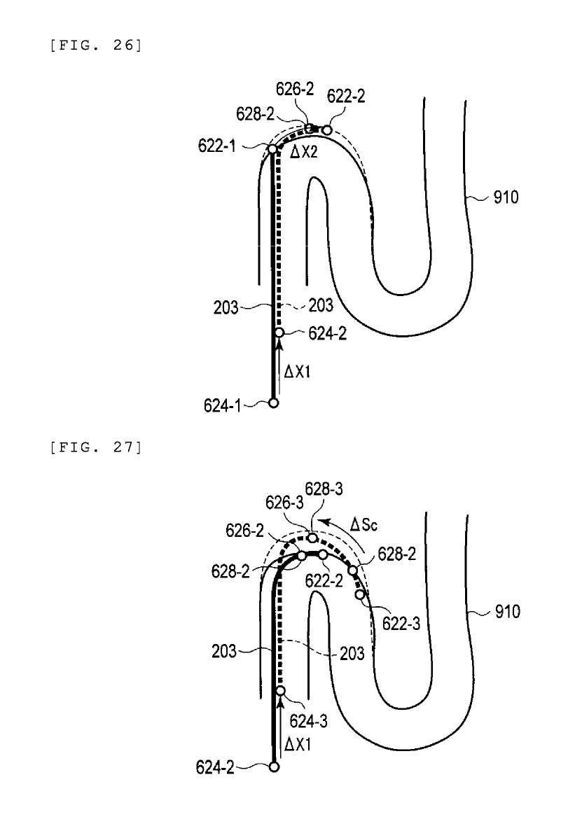

Meanwhile, FIG. 28 schematically illustrates the shape of the insertion portion 203 at the time point t2 and the time point t3 in a case where the insertion portion 203 is not inserted along the subject 910. Also in this case, the rear-side detection point of the insertion portion 203 is inserted by the distance ΔX1. In the case illustrated in FIG. 28, the insertion portion 203 is in the stick state and the subject 910 is extended.

When the folding end of the region, in which the insertion portion 203 bends at the time point t3, is set as an attention point 626-3′, a point on the insertion portion 203, which is coincident with the attention point 626-3′, is set as a third point 628-3′. The point on the insertion portion 203 which represents the position of the attention point 626 moves by ΔSc in the rearward direction along the insertion portion 203 from the second point 628-2 to the third point 628-3′.

When the insertion portion 203 is not inserted along the subject, the point on the insertion portion 203, which represents the position of the attention point 626, changes from the second point 628-2 to the third point 628-3′, and the displacement ΔSc' thereof is smaller than the displacement ΔX1 of the rear-side detection point of the insertion portion 203.

As described above, the determination of whether or not the insertion portion 203 is inserted along the subject 910 can be performed, depending on an inserting amount of the insertion portion 203 and the change in the position of the attention point on the insertion portion 203. As described above, when the inserting amount of the insertion portion 203 is interlocked with the change in the position of the attention point on the insertion portion 203, the insertion portion 203 is clearly known to be inserted along the subject 910. When the inserting amount of the insertion portion 203 is not interlocked with the change in the position of the attention point on the insertion portion 203, the insertion portion 203 is clearly known not to be inserted along the subject 910.

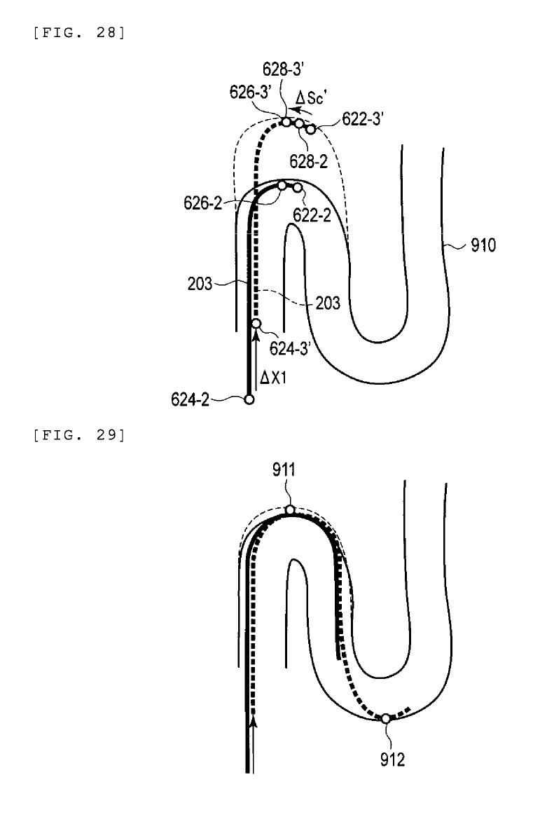

Similar to FIG. 27, FIGS. 29 and 30 further illustrate an example of a state obtained after the insertion portion 203 is inserted along the subject 910. FIG. 29 illustrates a case where the insertion portion 203 is inserted along the subject 910 in a first bending region 911 of the subject 910, which is illustrated on the upper side in FIG. 29, and the front end of the insertion portion 203 reaches a second bending region 912 of the subject 910, which is illustrated on the lower side in FIG. 29. FIG. 30 illustrates a case where the insertion portion 203 is inserted along the subject 910 in the first bending region 911; however, the insertion portion 203 is not inserted along the subject 910 in the second bending region 912, but the insertion portion 203 is in the stick state.

In the case illustrated in FIGS. 29 and 30, FIG. 31 schematically illustrates a change in the position of the attention point on the insertion portion 203. When time elapses in the order of the time points t1, t2, t3, and t4, and the insertion portion 203 is gradually inserted from the insertion opening of the subject 910, a first attention point R1 corresponding to the first bending region 911, which is first detected, moves toward the rear end side depending on the inserting amount.

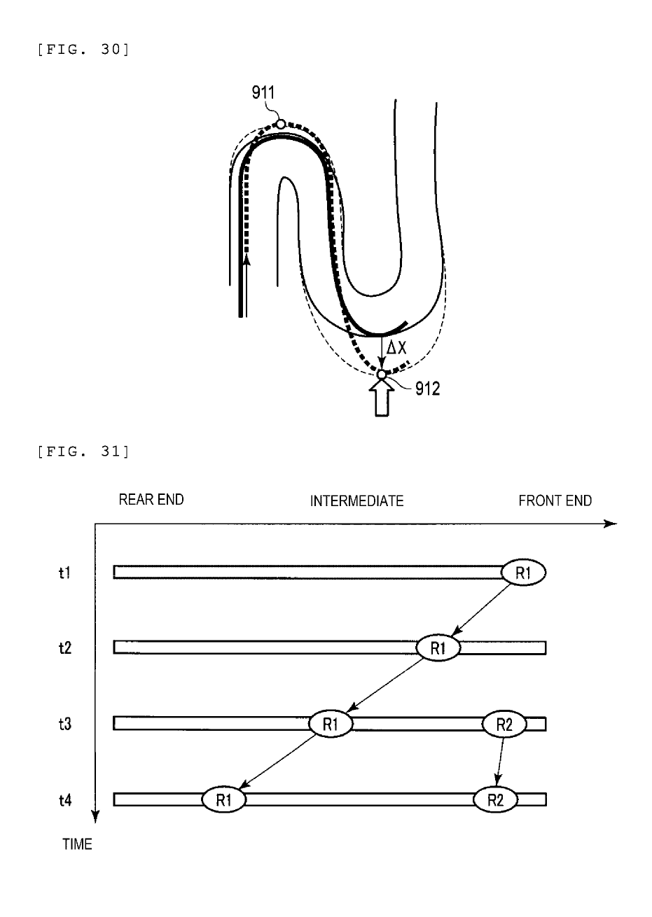

As illustrated in FIG. 31, a second attention point R2 corresponding to the second bending region 912 is detected at the time point t3. The second attention point R2 does not move toward the rear end side of the insertion portion 203 depending on the inserting amount. In addition, at this time, the shape of the insertion portion 203 at the second attention point R2 can change into the previous shape thereof. AS described above, in regions in which the self-compliance property is high and low, states of the changes in the position on the insertion portion 203, which corresponds to the point determined, based on the attention point, are different from each other.

The third state determination method is described with reference to FIGS. 32 to 35. As illustrated in FIG. 32, the insertion portion 203 transitions in the order of a first state 203-1, a second state 203-2, to a third state 203-3, as time elapses. A case, in which the insertion portion 203 is inserted along the subject 910 from the first state 203-1 to the second state 203-2, and the subject 910 is pressed by the insertion portion 203 and is extended toward the top point side from the second state 203-2 to the third state 203-3, is considered.

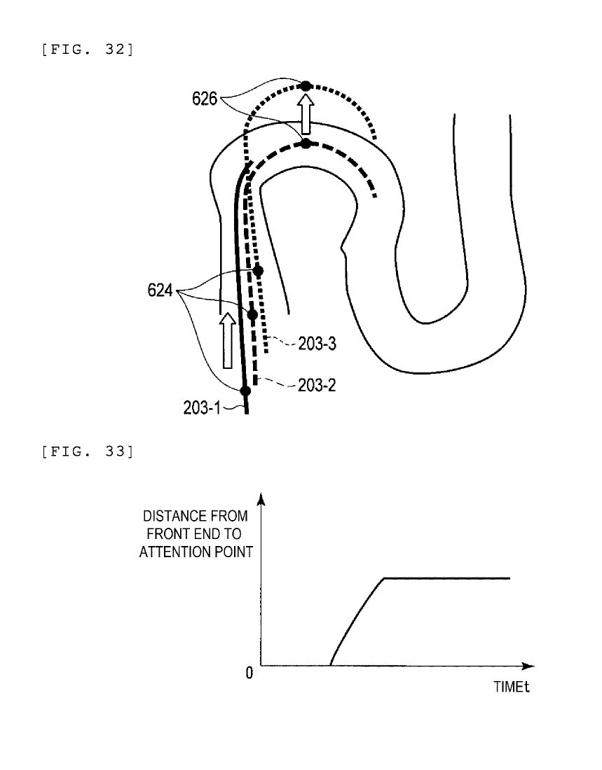

In such a case, in FIG. 33, the horizontal axis represents time elapse, that is, the displacement of a detection point 624 on the rear end side, and the vertical axis represents the position of the attention point 626 on the insertion portion 203, that is, the distance from the front end to the attention point 626. In other words, as illustrated in FIG. 33, the attention point is not detected for a short period from the start of the insertion as in the first state 203-1. When the insertion portion 203 is inserted along the subject 910 as between the first state 203-1 and the second state 203-2, the distance from the front end to the attention point gradually increases as illustrated in FIG. 33. When the insertion portion 203 is in the stick state as between the second state 203-2 to the third state 203-3, the distance from the front end to the attention point does not change as illustrated in FIG. 33.

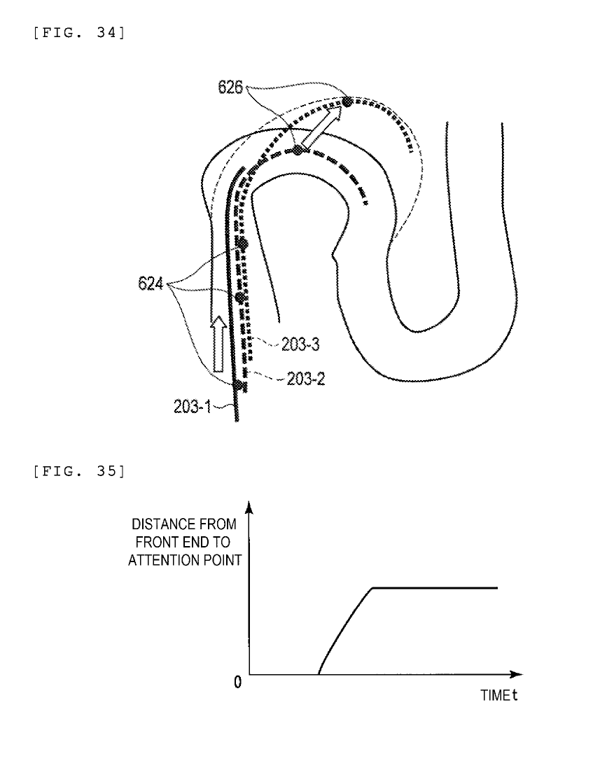

In addition, as illustrated in FIG. 34, a case, in which the insertion portion 203 is inserted along the subject 910 from the first state 203-1 to the second state 203-2, and the subject is pressed in an inclined direction from the second state 203-2 to the third state 203-3, is considered. Also in this case, similar to the case in FIG. 33, in FIG. 35, the horizontal axis represents the time elapse, that is, the displacement of a detection point 624 on the rear end side, and the vertical axis represents the position of the attention point 626 on the insertion portion 203, that is, the distance from the front end to the attention point 626.

When the movement amount of the attention point along the shape of the insertion portion 203 is set as ΔSc, and the moving amount of the detection point at an arbitrary spot of the insertion portion 203 on the rear end side is set as ΔX1, a determination expression representing a self-compliance property R is defined in the following expression.

R≡|ΔSc|/|ΔX1|

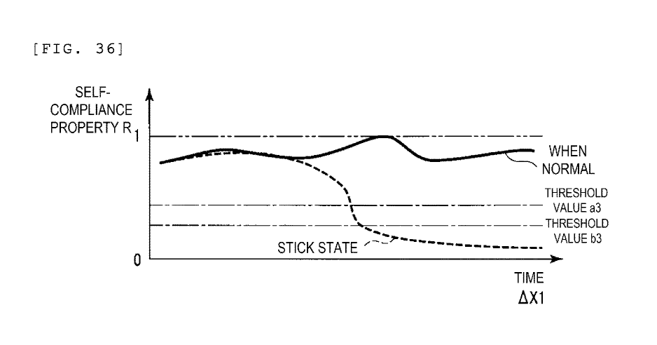

At this time, when the horizontal axis represents the time elapse or the moving amount ΔX1, that is, the inserting amount, of the corresponding arbitrary spot, and the vertical axis represents the self-compliance property R, a relationship illustrated in FIG. 36 is formed. In other words, when the insertion portion 203 is normally inserted along the subject, the self-compliance property R is an approximate value to 1 as represented by a solid line. Meanwhile, in the stick state, the self-compliance property R is a value smaller than 1 as represented by a dashed line.

The determination expression representing the self-compliance property R may be defined in the following expression.

R≡(ΔSc+C2)^{L}/(|ΔX1|+C1)^{M}

Here, C1, C2, L, and M are arbitrary real numbers, respectively.

For example, in a case where detected noise component levels of ΔX1 and ΔSc are N1 and Nc (N1 and Nc≧0), parameter C1·C2·L·M is set as follows.

C1=N1|ΔX1|≧N1

C2=−Nc|ΔX2|≧Nc

=−|ΔX2||ΔX2|<Nc

L=M=4

For example, N1 or Nc may be set to the value of about three times the standard deviation (σ) of the noise level.

Setting in which C1 is positive and C2 is negative is performed against noise as described above, thereby reducing the effect of the detection noise, and the self-compliance property R as the manipulation support information, with which false detection due to the detection noise is lowered, is obtained. In addition, a degree of L·M is a value of 2 or higher, thereby a ratio of ΔSc to ΔX1 sensitively decreases, and it is likely to determine degradation of the self-compliance property. In addition, a method of reducing the noise effect can also be applied to a case of other support information calculations.

As illustrated in FIG. 36, regarding the self-compliance property R, it is possible to appropriately set threshold values, such as a threshold value a3 that is set as a value indicating that a warning that the subject 910 starts to be extended needs to be output, and a threshold value b3 that is set as a value indicating that a warning that there is a danger to the subject, if the subject 910 is further extended, needs to be output. Appropriate setting of the threshold value enables the self-compliance property R to be used as information for supporting the manipulation of the endoscope 200, such as an output of warning to a user or a warning signal to the control device 310.

FIG. 37 schematically illustrates an example of a configuration of the manipulation support device for executing the third state determination method.

The insertion-extraction support device 100 includes the position acquiring unit 110, the shape acquiring unit 120, the state determination unit 130, and the support information generating unit 180. The detection point acquiring unit 111 of the position acquiring unit 110 obtains, for example, the position of the detection point as the spot of the insertion portion 203 on the rear end side, at which the position sensor is disposed, based on the information output from the sensor 201.

The shape acquiring unit 120 obtains the shape of the insertion portion 203, based on the information output from the sensor 201. The attention point acquiring unit 121 of the shape acquiring unit 120 obtains the position of the attention point, based on the shape of the insertion portion 203.

The state determination unit 130 includes a displacement acquiring unit 161, a displacement information calculation unit 162, and an attention point state determination unit 163. The displacement acquiring unit 161 calculates the displacement of the position on the insertion portion 203 of the attention point, based on the shape of the insertion portion 203, the position of the attention point, and displacement analysis information 192-5 recorded in the program memory 192. In addition, the displacement acquiring unit 161 calculates the displacement of the position of the detection point, based on the position of the detection point of the insertion portion 203 on the rear end side, and the displacement analysis information 192-5 recorded in the program memory 192. As described above, the displacement acquiring unit 161 functions as the first displacement acquiring unit that obtains the first displacement of the attention point, and further functions as the second displacement acquiring unit that obtains the second displacement of the detection point.

The displacement information calculation unit 162 calculates the displacement information in comparison of the displacement of the attention point on the insertion portion 203 with the displacement of the detection point of the insertion portion 203 on the rear end side, using the displacement analysis information 192-5 recorded in the program memory 192. The attention point state determination unit 163 calculates a state of the attention point, based on the displacement information and determination reference information 192-6 recorded in the program memory 192.

The support information generating unit 180 generates the manipulation support information, based on the determined state of the attention point. The manipulation support information is subjected to feedback in control by the control device 310, is displayed on the display device 320, or is recorded in the recording device 196.

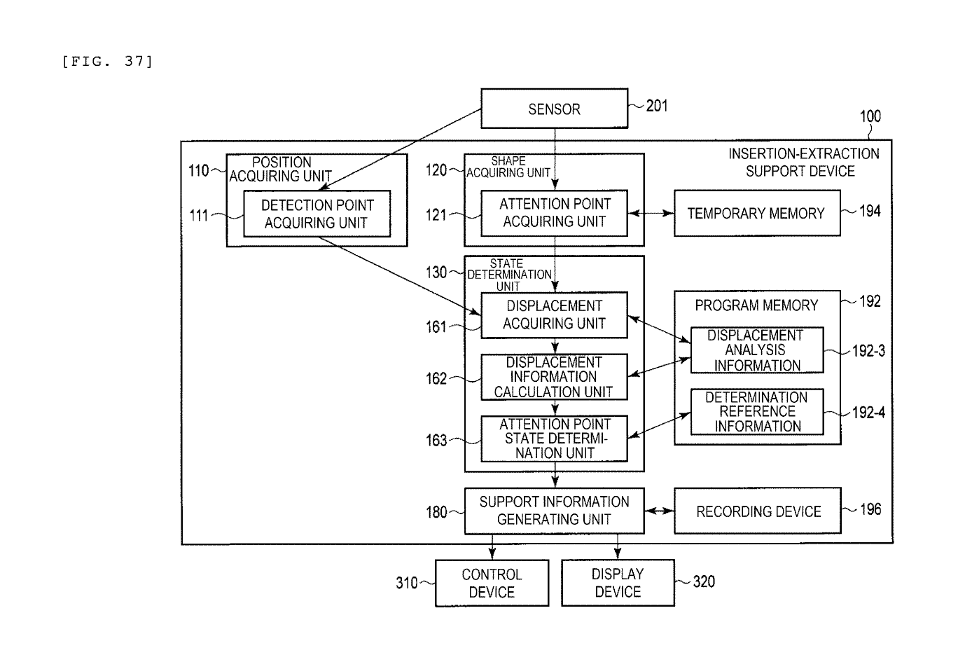

The operation of the insertion-extraction support device 100 in the third state determination method is described with reference to a flowchart illustrated in FIG. 38.

In Step S301, the insertion-extraction support device 100 acquires the output data from the sensor 201. In Step S302, the insertion-extraction support device 100 obtains the position of the detection point on the rear end side, based on the data acquired in Step S301.

In Step S303, the insertion-extraction support device 100 obtains the shape of the insertion portion 203, based on the data acquired in Step S301. In Step S304, the insertion-extraction support device 100 obtains the position of the attention point, based on the shape of the insertion portion 203 obtained in Step S303.

In Step S305, the insertion-extraction support device 100 calculates the position of the attention point on the insertion portion 203. In Step S306, the insertion-extraction support device 100 acquires successive changes in the position of the attention point on the insertion portion 203. In Step S307, the insertion-extraction support device 100 calculates an evaluation value of the positional change in the attention point on the insertion portion 203 with respect to the self-compliance property R or like, based on the positional change in the detection point and the positional change in the attention point on the insertion portion 203. In Step S308, the insertion-extraction support device 100 evaluates the extension such as whether or not the extension of the subject occurs or what a degree the extension occurs on the periphery of the attention point, based on the evaluation value calculated in Step S307.

In Step S309, the insertion-extraction support device 100 generates appropriate support information that is used in the following processes, based on the determination results of whether or not the extension of the subject occurs, the self-compliance property R, or the like, and outputs the support information, for example, to the control device 310 or to the display device 320.

In step S310, the insertion-extraction support device 100 determines whether or not the end signal for ending the processes has been input. When the end signal is not input, the process returns to Step S301. In other words, the processes described above are repeated until the end signal is input and the manipulation support information is output. On the other hand, when the end signal is input, the corresponding process is ended.

The third state determination method is used, thereby the displacement of the attention point on the insertion portion 203 is identified, and the manipulation support information indicating whether or not the extension occurs in the subject can be generated, based on the displacement and the inserting amount of the insertion portion 203 on the rear end side, that is, a relationship between the displacements of the detection points, or the like. The manipulation support information includes, for example, the state of the insertion portion 203 or the subject 910, presence or absence of the pressure or compression of the insertion portion 203 with respect to the subject 910, a magnitude thereof, or the like. In addition, the manipulation support information includes information associated with whether or not the abnormality occurs in the insertion portion 203 or the subject 910.

Similar to the attention point used in the second state determination method, the attention point used in the third state determination method may be disposed at any position as long as the position is determined, based on the shape of the insertion portion 203. For example, the attention point may be the folding end of the bending region as in the embodiment described above, may be the bending start position of the bending region, may be any position in a straight line-shaped region, for example, as an intermediate point between the bending region and the front end, or may be an intermediate point or the like between a bending region and another bending region in the case where two or more bending regions occur. In addition, the position of the detection point is not limited to the rear end side, and may also be any position. In addition, instead of the detection point, the attention point as an arbitrary spot may be used. In a case where the position of the attention point is used, the detection point acquiring unit 111 does not obtain the positions, but the position acquiring unit 110 obtains the positions of the attention points, and the obtained positions of the attention points are used.

In a modification example of the third state determination method, the state of the insertion portion 203 is determined, based on the moving amount of the insertion portion 203 in a tangential direction of the shape of the insertion portion 203. In particular, the state of the insertion portion 203 is determined, based on the moving amount of the insertion portion 203 in the tangential direction at the attention point.

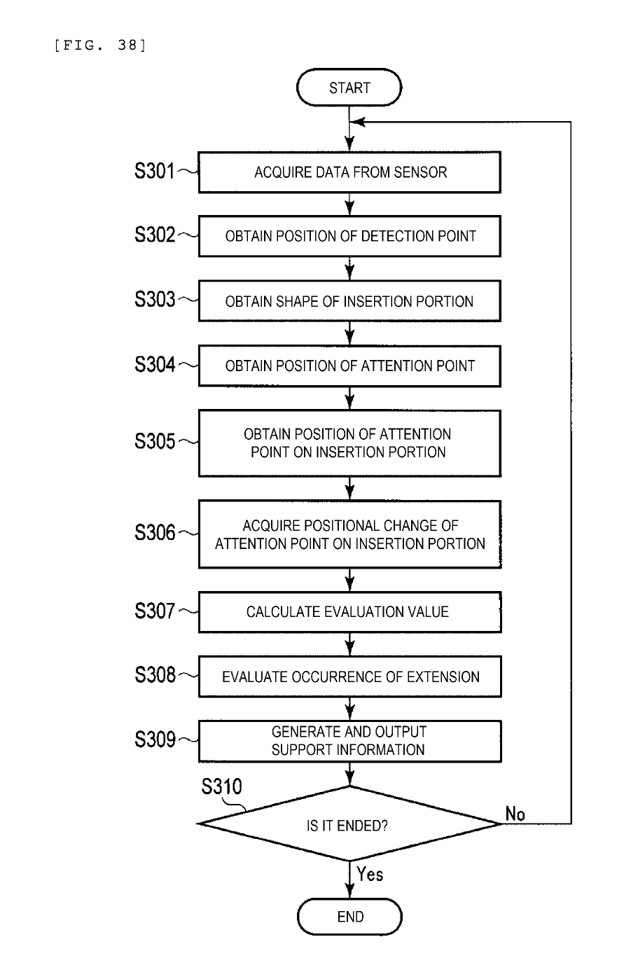

As schematically illustrated in FIG. 39, an attention point 631 is acquired, based on the shape of the insertion portion 203. Subsequently, a tangential direction 632 of the insertion portion 203 at the attention point 631 is identified, based on the shape of the insertion portion 203. In the modification example of the third state determination method, the self-compliance property is evaluated, based on a relationship between a moving direction of a point on the insertion portion 203, which corresponds to the attention point 631, and the tangential direction 632. In other words, it turns out that the more the moving direction of the point on the insertion portion 203, which corresponds to the attention point 631, is coincident with the tangential direction 632 of the insertion portion 203, the higher the self-compliance property.

As illustrated in FIG. 40, the state of the insertion portion 203 or the state of the subject 910 is evaluated, for example, based on a ratio of a displacement amount ΔSr in the tangential direction of a displacement amount ΔX to the displacement amount ΔX of the point corresponding to the attention point. In other words, the state of the insertion portion 203 or the state of the subject 910 is evaluated, based on an angle θ formed between the tangential direction and the moving direction at the attention point.

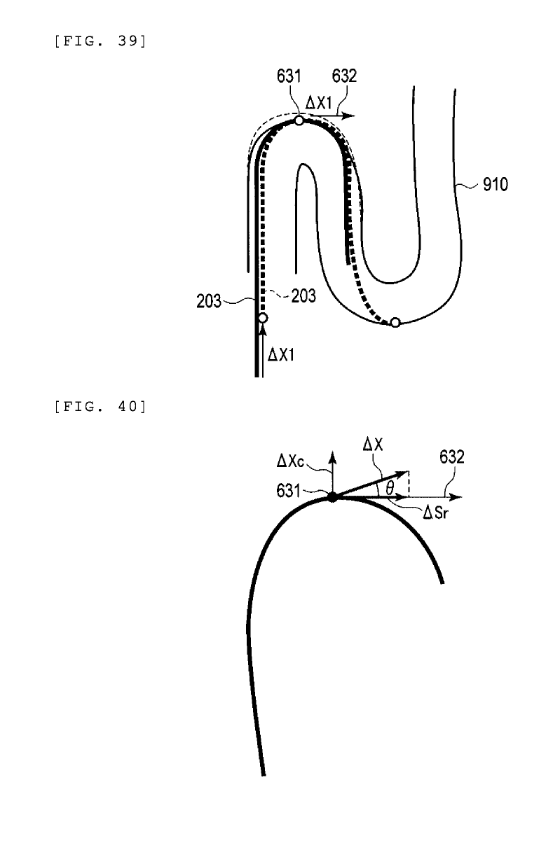

As illustrated in FIG. 32 described above, the insertion portion 203 transitions in the order of the first state 203-1, the second state 203-2, to the third state 203-3, as time elapses. In such a case, |ΔSr|/|ΔX| representing the ratio of the displacement in the tangential direction to the displacement of the insertion portion 203 with respect to the time elapse is illustrated in FIG. 41. Since the self-compliance property is high between the first state 203-1 and the second state 203-2, the ratio of the displacement in the tangential direction with respect to the moving direction of the point to the displacement of the insertion portion 203 is substantially 1. Meanwhile, since the insertion portion 203 does not move in the tangential direction, but displacements while causing the subject 910 to be extended in a direction perpendicular to a tangential line from the second state 203-2 to the third state 203-3, the ratio of the displacement in the tangential direction with respect to the moving direction of the point to the displacement of the insertion portion 203 is substantially 0.

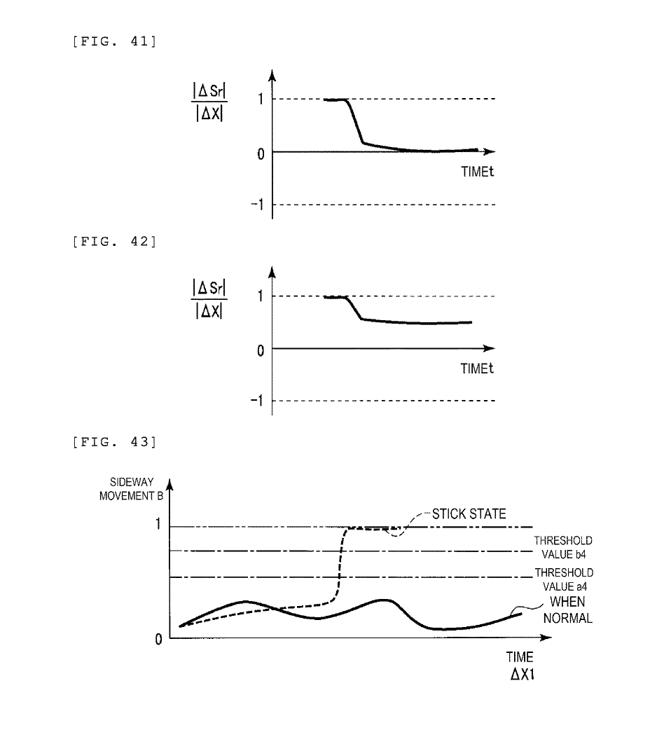

As illustrated in FIG. 34 described above, the insertion portion 203 transitions in the order of the first state 203-1, the second state 203-2, to the third state 203-3, as time elapses. In such a case, |ΔSr|/|ΔX| in the displacement of the insertion portion 203 with respect to the time elapse is illustrated in FIG. 42. Since the self-compliance property is high between the first state 203-1 and the second state 203-2, the ratio of the displacement in the tangential direction with respect to the moving direction of the point to the displacement of the insertion portion 203 is substantially 1. Meanwhile, since the insertion portion 203 moves in a direction inclined with respect to the tangential direction from the second state 203-2 to the third state 203-3, the ratio of the displacement in the tangential direction with respect to the moving direction of the point to the displacement of the insertion portion 203 is substantially 0.5.

Note that, in a case where ΔSr and ΔX are vectors, (ΔSr·ΔX)/(|ΔSr|×|ΔX|) or cos θ may be used as an index. In this manner (“·” representing a dot product), the self-compliance property turns out to be very low in a case where ΔX and ΔSr represent shifts in opposite directions, compared to a case where the self-compliance property is verified simply using |ΔSr|/|ΔX|.

In the description of the modification example of the third state determination method described above, the value used in the evaluation is described as the movement of the point on the insert in the tangential direction, which corresponds to the attention point; however, the value may be evaluated as the movement in a direction perpendicular to the tangential line, that is, the movement of the insertion portion 203 in a horizontal direction. For example, when the movement amount of the attention point in the direction perpendicular to the tangential line of the insertion portion 203 is set as ΔXc as illustrated in FIG. 40, and the moving amount of the attention point or the detection point at an arbitrary spot of the insertion portion 203 on the rear end side is set as ΔX1, a determination expression representing a sideway movement B is defined in the following expression.

B=|ΔXc|/|ΔX1|

At this time, when the horizontal axis represents the time elapse or the moving amount ΔX1, that is, the inserting amount, of the corresponding arbitrary spot, and the vertical axis represents the sideway movement B, a relationship illustrated in FIG. 43 is formed. In other words, when the insertion portion 203 is normally inserted along the subject, the sideway movement B is an approximate value to 0 as represented by a solid line. Meanwhile, in the stick state, the sideway movement B is an approximate value to 1 as represented by a dashed line.

As illustrated in FIG. 43, regarding the sideway movement B, it is possible to appropriately set threshold values, such as a threshold value a4 that is set as a value indicating that a warning that the subject 910 starts to be extended needs to be output, and a threshold value b4 that is set as a value indicating that a warning that there is a danger to the subject, if the subject 910 is further extended, needs to be output. Appropriate setting of the threshold value enables the sideway movement B to be used as information for supporting the manipulation of the endoscope 200, such as an output of a warning to a user or a warning signal to the control device 310.

A movement of a point of the insertion portion 203, to which attention is paid, may be described as the sideway movement, may be described as the movement in the tangential direction, or may be described in any manner. The meaning is the same. In addition, in any case, a moving amount of a point, to which attention is paid, may be compared to a moving amount of the attention point or the detection point of the insertion portion 203 on the rear end side, or analysis may be performed, based on only a ratio of a component of the movement in the tangential direction to the movement of the point to which the attention is paid, without using the moving amount of the attention point or the detection point on the rear side. In addition, in any case, it turns out that the higher the tangential direction of the insertion portion 203 is coincident with the moving direction of the insertion portion, the higher self-compliance property the movement of the insertion portion 203 has, such that the insertion portion 203 is inserted along the subject 910. In this respect, the same is true in the following description.

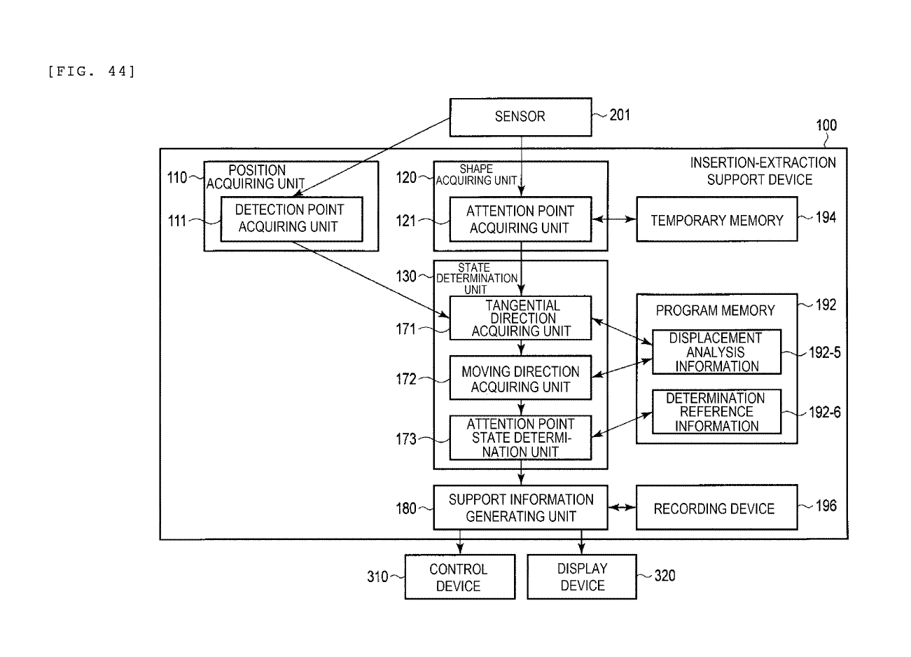

FIG. 44 schematically illustrates an example of a configuration of the manipulation support device for executing a fourth state determination method. Here, an example of the configuration of the manipulation support device, in the case where the detection point on the rear end side is used, is described.

The insertion-extraction support device 100 includes the position acquiring unit 110, the shape acquiring unit 120, the state determination unit 130, and the support information generating unit 180. The detection point acquiring unit 111 of the position acquiring unit 110 obtains, for example, the position of the detection point as the spot of the insertion portion 203 on the rear end side, at which the detection of the position is performed, based on the information output from the sensor 201.

The shape acquiring unit 120 obtains the shape of the insertion portion 203, based on the information output from the sensor 201. The attention point acquiring unit 121 of the shape acquiring unit 120 obtains the position of the attention point.

The state determination unit 130 includes a tangential direction acquiring unit 171, a moving direction acquiring unit 172, and an attention point state determination unit 173. The tangential direction acquiring unit 171 calculates the tangential direction of the insertion portion 203 at the attention point, based on the shape of the insertion portion 203, the position of the attention point, and displacement analysis information 192-5 recorded in the program memory 192. The moving direction acquiring unit 172 calculates the moving direction of the attention point, based on the position of the attention point, and the displacement analysis information 192-5 recorded in the program memory 192. The attention point state determination unit 173 calculates the state of the attention point, based on the tangential direction of the attention point on the insertion portion 203, the moving direction of the attention point, and the determination reference information 192-6 recorded in the program memory 192.

The support information generating unit 180 generates the manipulation support information, based on the determined state of the attention point. The manipulation support information is subjected to feedback in control by the control device 310, is displayed on the display device 320, or is recorded in the recording device 196.

The operation of the insertion-extraction support device 100 in the fourth state determination method is described with reference to a flowchart illustrated in FIG. 45.

In Step S401, the insertion-extraction support device 100 acquires the output data from the sensor 201. In Step S402, the insertion-extraction support device 100 obtains the position of the detection point on the rear end side, based on the data acquired in Step S401.

In Step S403, the insertion-extraction support device 100 obtains the shape of the insertion portion 203, based on the data acquired in Step S401. In Step S404, the insertion-extraction support device 100 obtains the position of the attention point, based on the shape of the insertion portion 203 obtained in Step S403.

In Step S405, the insertion-extraction support device 100 calculates the tangential direction of the insertion portion 203 at the attention point. In Step S406, the insertion-extraction support device 100 obtains the moving direction of a position of the insertion portion 203, which corresponds to the attention point, and calculates a value representing the sideway movement.

In Step S407, the insertion-extraction support device 100 calculates an evaluation value representing the self-compliance property R at the attention point of the insertion portion 203, based on the positional change in the detection point and the value representing the sideway movement. The smaller the value representing the sideway movement with respect to the positional change in the detection point, the higher the self-compliance property.

In Step S408, the insertion-extraction support device 100 evaluates the extension such as whether or not the extension of the subject occurs or what a degree the extension occurs on the periphery of the attention point, based on the evaluation value calculated in Step S407.

In Step S409, the insertion-extraction support device 100 generates appropriate support information that is used in the following processes, based on the determination results of whether or not the extension of the subject occurs, and outputs the support information, for example, to the control device 310 or to the display device 320.

In step S410, the insertion-extraction support device 100 determines whether or not the end signal for ending the processes has been input. When the end signal is not input, the process returns to Step S401. In other words, the processes described above are repeated until the end signal is input and the manipulation support information is output. On the other hand, when the end signal is input, the corresponding process is ended.

The fourth state determination method is used, and thereby the manipulation support information indicating whether or not the extension occurs in the subject can be generated, based on the relationship between the moving direction and the tangential direction at the attention point on the insertion portion 203. The manipulation support information can include, for example, the state of the insertion portion 203 or the subject 910, presence or absence of the pressure or compression of the insertion portion 203 with respect to the subject 910, a magnitude thereof, or presence or absence of abnormality of the insertion portion 203.

Note that, in the example described above, the case where the analysis is performed with the attention point as a target is described; however, the analysis target is not limited thereto. Instead of the attention point, the self-compliance property can be evaluated at an arbitrary point, based on the tangential direction at the point, which is obtained from the shape thereof, and the moving direction of the point.

In addition, In the description provided above, the example, in which the self-compliance property is evaluated, based on the relationship between the moving amount of the detection point of the insertion portion 203 on the rear end side and the moving amount of the attention point, is provided. Instead of the detection point, an arbitrary attention point may be used. In addition, the moving amount of the detection point does not need to be necessarily considered. In addition, regarding the moving amount of the attention point, the self-compliance property can be evaluated, also based on only the ratio of the component in the direction perpendicular to the tangential line to a component in the tangential direction.

Note that the third state determination method and the fourth state determination method are common in that the self-compliance property of the insertion portion 203 is evaluated.

In the description provided above, an example in which the movement of the attention point in the tangential direction is analyzed, based on the shape of the insertion portion 203. The analysis is not limited to the attention point, the movement of the front end of the insertion portion 203 in the tangential direction may be analyzed. The tangential direction of the front end means, that is, a direction in which the front end of the insertion portion 203 faces forward.

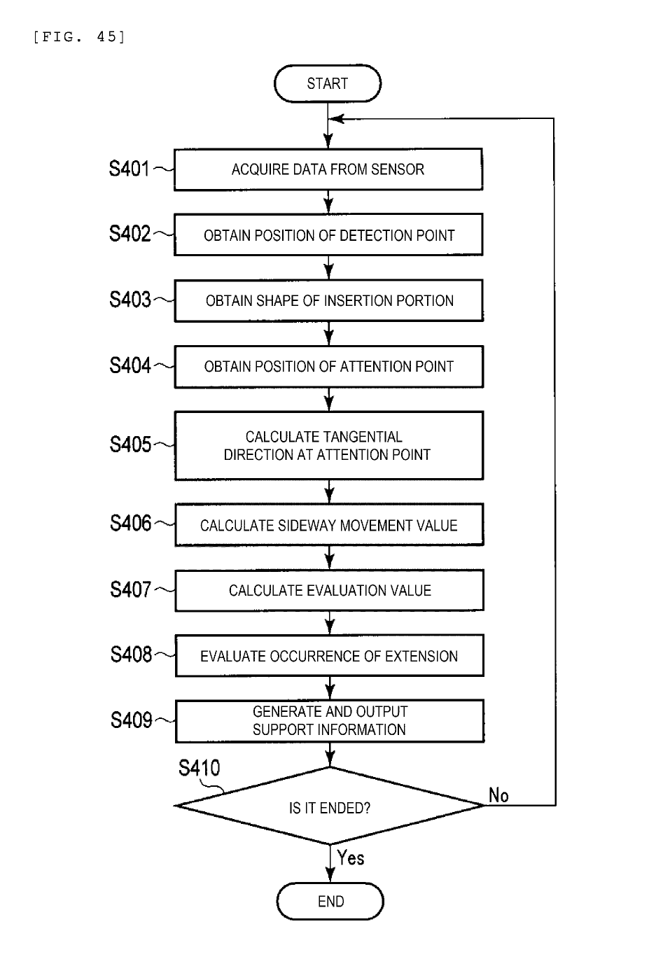

In the same state as illustrated in FIG. 32, as illustrated in FIG. 46, the front end of the insertion portion 203 moves in the rearward direction from the second position 635-2 to the third position 635-3. In other words, return of the front end occurs. In a case where the endoscope 200 is an endoscope that acquires an image in a front end direction, it is possible to find the movement of the front end of the insertion portion 203 in the rearward direction, based on the acquired image.

front end advance P representing an advance condition of the front end portion of the insertion portion 203 in the front end direction is defined in the following expression.

P=(ΔX2·D)/|ΔX1|

Here, ΔX2 represents a displacement vector of the front end, D represents a vector in the front end direction, and “·” represents a dot product.

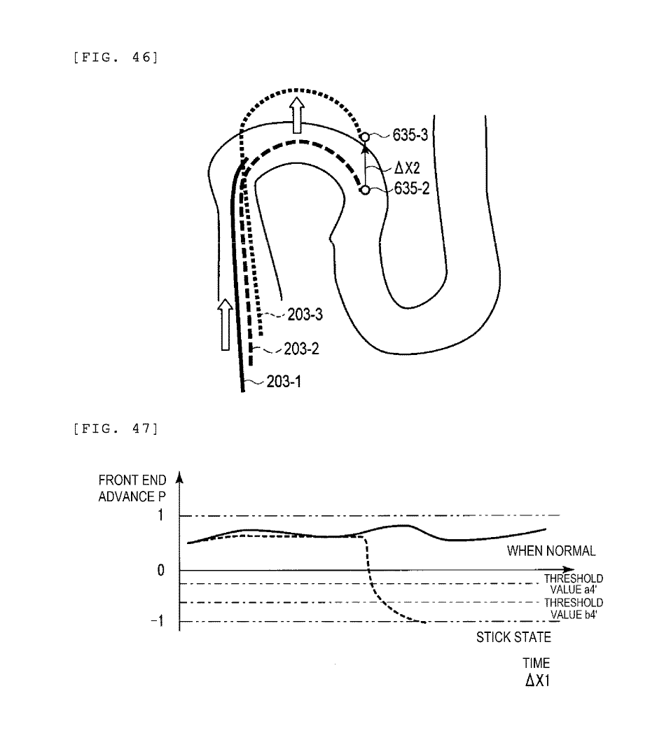

FIG. 47 illustrates an example of a change in the front end advance P with respect to the time elapse, that is, the inserting amount ΔX1 at an arbitrary spot on the rear end side. The solid line in FIG. 47 represents a case where the insertion portion 203 is inserted along the subject 910. In this case, since the front end of the insertion portion 203 advances in the front end direction, a value of the front end advance P is an approximate value to 1. On the other hand, the dashed line in FIG. 47 represents a case where the insertion portion 203 is in the stick state. In this case, since the front end portion of the insertion portion 203 advances in the rearward direction, the front end advance P is an approximate value to −1.

As illustrated in FIG. 47, regarding the front end advance P, it is possible to appropriately set threshold values, such as a threshold value a4′ that is set as a value indicating that a warning that the subject 910 starts to be extended needs to be output, and a threshold value b4′ that is set as a value indicating that a warning that there is a danger to the subject, if the subject 910 is further extended, needs to be output. Appropriate setting of the threshold value enables the front end advance P to be used as information for supporting the manipulation of the endoscope 200, such as an output of a warning to a user or a warning signal to the control device 310.

As described above, the state of the insertion portion 203 or the subject 910 can be determined with the front end advance P which is characteristically detected as the return of the front end.

The state determination methods described above all evaluate a degree of the self-compliance property. A state in which there is a difference between the moving amounts of the two or more attention points can also be described, in other words, as a state in which there is a spot between the two points, at which the self-compliance property is low. In addition, the stick state can be described, in other words, as a state in which the sideway movement occurs, and the sideway movement can also be described, in other words, as a state in which the self-compliance property is low.

In the first state determination method, when detection of a difference between the moving amounts of the two or more attention points is performed, and the difference is detected, for example, determination that buckling occurs is performed. When the buckling occurs, a state in which the self-compliance property is low at the spot at which the buckling occurs is detected.

In the second state determination method, the attention is paid to the attention point, and a state in which there is no self-compliance property in the bending region, that is, a state in which the sideway movement occurs in the bending region and the subject 910 is pushed upward is detected.

In the third state determination method, the attention is paid to the attention point, and the self-compliance property is evaluated, based on the position of the attention point on the insertion portion 203. When the self-compliance property is high, the self-compliance property is evaluated, using a state in which the distance of the position of the attention point on the insertion portion 203 is coincident with the inserting amount.

In the fourth state determination method, the self-compliance property is evaluated, based on a tangential line at a certain point and a moving direction of the point. When the self-compliance property is high, the self-compliance property is evaluated, using a state in which a predetermined point advances in the tangential direction of the shape of the insertion portion 203 at the point. On the other hand, when the self-compliance property is low, for example, the sideway movement or the like occurs.

In addition, the state in which the self-compliance property is low can be described, in other words, as the state in which the sideway movement occurs. Hence, the state determination methods described above can all be described, in other words, as a method in which a degree of the sideway movement is evaluated, or can be described to be the same.

Here, there is a region in which the subject bends, as a spot on the insertion portion 203 or the subject 910, to which attention is paid. In the region which bends, since the self-compliance property of the insertion portion 203 is lowered, and a wall of the subject is pressed when the sideway movement occurs in the bending region, the evaluation value is high in the state of the insertion portion 203 or the subject 910 in the bending region of the subject. Thus, in the second state determination method, the third state determination method, and the fourth state determination method, attention is paid to the bending region as the attention point and analysis is performed on the bending region.

However, the attention point is not limited thereto, and by the same method, various spots can be set as the attention point, and the states of the insertion portion 203 or the subject 910 at the various spots are analyzed.

As described above, the displacement information acquiring unit 141 and the interlocking condition calculation unit 142, the displacement acquiring units 151 and 161 and the displacement information calculation units 152 and 162, or the tangential direction acquiring unit 171 and the moving direction acquiring unit 172 function as a self-compliance property evaluating unit that evaluates the self-compliance property in the insertion of the insertion portion 203. In addition, the buckling determination unit 143 or the attention point state determination units 153, 163, and 173 function as a determination unit that determines the state of the insertion portion 203 or the subject 910, based on the self-compliance property.

The state of the insertion portion 203 or the subject 910 is used in the determination of whether or not the insertion portion 203 is inserted along the subject 910. When the insertion portion 203 is inserted into the subject 910, a user intentionally changes the shape of the subject. For example, in the region in which the subject 910 bends, the shape of the subject is manipulated to be close to a straight line such that the insertion portion 203 is likely to advance. Also in such a manipulation, information associated with the shape of the insertion portion 203, the shape of the subject 910, a force applied to the subject 910 by the insertion portion 203, or the like is useful information for the user.

The first to fourth state determination methods can be combined to be used. For example, the first state determination method and another state determination method are combined to be used, and thereby the following effects are achieved. In other words, the use of the first state determination method makes it possible to acquire information associated with the buckling which occurs in the insertion portion 203. A component of the displacement derived from the buckling is subtracted, and thereby it is possible to improve accuracy of the calculation results by the second to fourth state determination methods, and it is possible to find phenomena which occur in the insertion portion 203 with accuracy. Besides, when the first to fourth state determination methods are used, an amount of acquired information increases, compared to a case where one method is used. This is effective to improve the accuracy of the generated support information.

The support information generating unit 180 generates the manipulation support information, using the first to fourth state determination methods and using the acquired information associated with the state of the insertion portion 203 or the subject 910. The manipulation support information is information for supporting the user who inserts the insertion portion 203 into the subject 910.

The manipulation support information can be generated, not only based on the information associated with the state of the insertion portion 203 or the subject 910, which is acquired using the first to fourth state determination methods, but also by combining various types of information such as information input from the input device 330 or information input from the control device 310. The first to fourth state determination methods are appropriately used, and thereby it is possible to appropriately acquire necessary information.

The manipulation support information is displayed, for example, on the display device 320, and the user performs the manipulation of the endoscope 200 with reference to the display. In addition, the manipulation support information is subject to the feedback in the control by the control device 310. More appropriate control of the operation of the endoscope 200 by the control device 310 supports the manipulation of the endoscope 200 by the user. The use of the manipulation support information enables the manipulation of the endoscope 200 to be smoothly performed.

Generation of the support information associated with the manipulation by the insertion-extraction support device 100 that functions as the manipulation support device is further described. FIG. 48 schematically illustrates an example of a configuration of a manipulation support information generating device 700 included in the insertion-extraction support device 100. The manipulation support information generating device 700 has functions of the position acquiring unit 110, the shape acquiring unit 120, the state determination unit 130, and the support information generating unit 180, which are described above. As illustrated in FIG. 48, the manipulation support information generating device 700 includes a manipulation support information generating unit 710, a use environment setting unit 730, a primary information acquiring unit 750, and a database 760.

The primary information acquiring unit 750 acquires primary information output from the sensor 201. The database 760 is recorded in a recording medium provided in the manipulation support information generating device 700. The database 760 includes information necessary for various operations of the manipulation support information generating device 700. The database 760 includes information necessarily used when information associated with setting that is determined particularly by the use environment setting unit 730 is derived.

The manipulation support information generating unit 710 acquires output information associated with the sensor 201 provided in the endoscope 200 via the primary information acquiring unit 750, generates high-order information while performing processing on the information, and finally generates the support information associated with the manipulation. Here, raw data output from the sensor 201 is referred to as the primary information. Information that is directly derived from the primary information is referred to as secondary information. Information that is derived from the primary information and the secondary information is referred to as tertiary information. Hereinafter, high-order information associated with fourth order information and fifth order information is derived by using low order information. As described above, the information processed in the manipulation support information generating unit 710 forms an information group having a hierarchy. In addition, items of information that belong to different hierarchies are different in a degree of the processing.

The manipulation support information generating unit 710 includes a secondary information generating unit 712, a high-order information generating unit 714, and a support information generating portion 716.

As described above, since the sensor 201 includes a plurality of sensors, the sensors are referred to as a first sensor 201-1, a second sensor 201-2, or the like. Note that the number of the sensors may not be limited to any number. The primary information acquiring unit 750 inputs the outputs from the sensor 201 such as the first sensor 201-1 or the second sensor 201-2 to the secondary information generating unit 712. The secondary information generating unit 712 generates the secondary information, based on the primary information acquired by the primary information acquiring unit 750. In the embodiment described above, for example, the detection point acquiring unit 111 of the position acquiring unit 110 functions as the secondary information generating unit 712. In addition, when the shape of the insertion portion 203 is calculated, based on the output of the shape sensor, a part of the shape acquiring unit 120 functions as the secondary information generating unit 712.

The high-order information generating unit 714 includes a tertiary order information generating unit or a fourth order information generating unit, which are not illustrated, and generates tertiary or higher order information. The high-order information is generated using low order information having a hierarchy lower than the corresponding information. In the example described above, a part of the position acquiring unit 110 and the shape acquiring unit 120 or the state determination unit 130 functions as the high-order information generating unit 714.

Here, the support information generating unit 716 corresponds to the support information generating unit 180, and generates support information associated with the manipulation, based on at least one item of the primary information, the secondary information generated by the secondary information generating unit 712, and the high-order information generated by the high-order information generating unit 714. The generated support information is output to the control device 310 or the display device 320.

As described above, in the manipulation support information generating unit 710, the information is converted from raw data acquired from the sensor 201 into a unit that a user can discern, further, is converted from the unit or the like that the user can discern into information that indicates states of the portions of the insertion portion 203, further, is converted from the information that indicates the states of the portions of the insertion portion 203 into insertion states of the insertion portion 203, and furthermore is converted from the insertion states of the insertion portion 203 into support information associated with the manipulation.

As described above, in the manipulation support information generating unit 710, a plurality of items of information belonging to a plurality of hierarchies are generated as the information group, and when the information included in the information group is defined as the state information, the support information associated with the manipulation can be generated based on a plurality of different items of the state information.

The use environment setting unit 730 analyzes a use environment, based on the information acquired from the endoscope 200, the input device 330, the recording device 196, or the like, and determines setting information necessary for the generation of the support information associated with the manipulation by the manipulation support information generating unit 710. The determined setting information is output to the manipulation support information generating unit 710. The manipulation support information generating unit 710 generates the support information associated with the manipulation, based on the setting information. Examples of the use environment described here include a type or performance of the endoscope 200, an environment in which the endoscope 200 is used or a state of the endoscope 200, a user who manipulates the endoscope 200 or proficiency of the user, the subject, an operative method, or the like.

The use environment setting unit 730 includes an environment determination unit 732, an information generation setting unit 742, and a setting criteria storage unit 744.

The environment determination unit 732 includes an insert information determination unit 734 and a user information determination unit 736. The insert information determination unit 734 acquires the output data of the sensor 201 via the primary information acquiring unit 750 from the sensor 201 of the endoscope 200. The insert information determination unit 734 determines the state of the endoscope 200, based on the output data of the sensor 201.

In addition, the endoscope 200 includes an identification information storage unit 282 in which identification information associated with the endoscope 200 is stored. Examples of the identification information include a model type and the serial number of the endoscope 200, information associated with a function or the like that the endoscope 200 has, a model type and the serial number of the sensor 201, information associated with a function or the like of the sensor 201, or the like. The insert information determination unit 734 acquires the identification information associated with the endoscope 200 from the identification information storage unit 282. The insert information determination unit 734 determines the state of the endoscope 200, based on the identification information associated with the endoscope 200. In addition, the insert information determination unit 734 specifies a combination between the insertion-extraction support device 100 and the endoscope 200, based on the identification information acquired from the identification information storage unit 282. The insert information determination unit 734 determines the support information which can be provided by the insertion-extraction support device 100, based on the combination.

The insert information determination unit 734 outputs, as insert-side information, the acquired information associated with the state of the endoscope 200 or the information associated with the providable support information, to the information generation setting unit 742.

The user information determination unit 736 acquires information that is input by a user by using the input device 330. In addition, the user information determination unit 736 acquires various items of information such as information associated with the user as a manipulator, the subject, and the like from the recording device 196, information associated with details of an operation performed using the endoscope 200, information associated with the endoscope 200 or the insertion-extraction support device 100, or information associated with the setting of the insertion-extraction support device 100. The information that is input by the user is referred to as first manipulator information. In addition, the information that is input from the recording device 196 is referred to as second manipulator information.

The user information determination unit 736 determines the user-side information, based on the acquired information. The user information determination unit 736 outputs the user-side information to the information generation setting unit 742. In addition, the user information determination unit 736 updates the information that is stored in the setting criteria storage unit 744 and the database 760 for the user-side information, as necessary.

The information generation setting unit 742 determines necessary setting for generating the high-order information or the support information associated with the manipulation by the manipulation support information generating unit 710, based on the insert-side information associated with the endoscope 200, which is acquired from the insert information determination unit 734, the user-side information associated with the user, which is acquired from the user information determination unit 736, the setting criteria information acquired from the setting criteria storage unit 744, and the information acquired from the database 760. The setting can include, for example, information associated with generated content of the support information associated with the manipulation, a method of generation, a timing of generation, or the like. For the determination of the setting, both of the insert-side information and the user-side information may be used, or either one may be used. The setting criteria storage unit 744 stores criteria information necessary for the setting performed by the information generation setting unit 742.

Here, information processed in the use environment setting unit 730 is described. The first manipulator information input by the user includes, for example, a request, determination, instruction, or the like from the manipulator.

An example of the first manipulator information is designation or the like of a method of providing a selection result of one or more items of support information that the user wants to use from the types of support information, or the selected support information. In addition, another example of the first manipulator information is a result or a reason of determination performed by the user based on images of the endoscope or the provided support information, or a method of coping with a phenomenon or an instruction to those involved, and is information that the manipulator inputs.

The input of the first manipulator information can be performed, for example, by using the pull-down menu displayed on the display device 320. Only providable support information is displayed as an option on the pull-down menu. The use of the pull-down menu enables to employ a configuration in which only the providable support information is selected. Note that a configuration in which the non-selectable support information is specified may be employed.

An example of the first manipulator information is described. Examples of a method of inserting a colonoscope include a loop method and an axis-holding shortening method. The loop method is a method of pushing and inserting the insertion portion 203 into the subject while the insertion portion 203 of the endoscope 200 forms a loop shape in a region where the intestine bends, and one of colonoscope inserting methods which have been used for a long time. The loop method is an inserting method in which the manipulation is easily performed for a doctor. Meanwhile, in the loop method, a patient is likely to have suffering when the loop is formed, and thus an analgesic is frequently used. On the other hand, the axis-holding shortening method is a colonoscope inserting method of directly inserting the insertion portion 203 of the endoscope 200 without forming the loop. In other words, in the axis-holding shortening method, a manipulator inserts the insertion portion 203 while carefully folding and shortening the intestine such that the intestine has a straight line shape. A doctor needs to have a skill to use the axis-holding shortening method; however, the patient has small suffering.

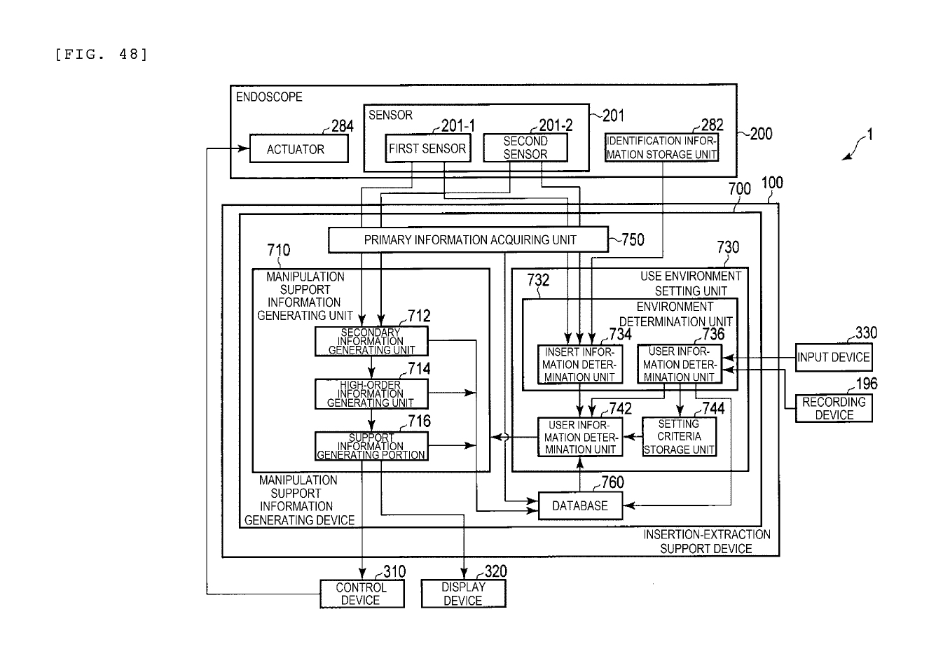

As the first manipulator information, for example, one of the loop method or the axis-holding shortening method is selected. FIG. 49 illustrates an example of menu items in this case. In FIG. 49, a lightly shaded item is, for example, an item that has been selected. In other words, the “manipulation support information” is selected in order to provide the support information associated with the manipulation, “insertion support” as one of the menu is selected, and “axis-holding shortening method” is selected from “axis-holding shortening method” and “loop method” as the menu.

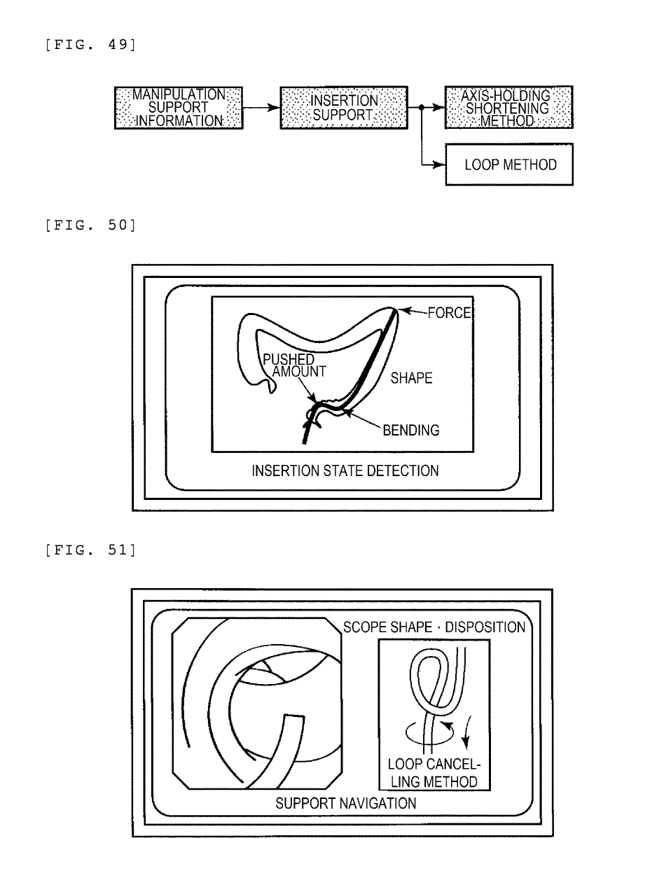

Another example of the first manipulator information includes the designation of the information that is considered to be particularly wanted by the manipulator. An example of the designated information includes the shape of the insertion portion 203 of the endoscope 200, instruction of inserting manipulation, or the like. The designated information is displayed on the display device 320 or the display thereof is highlighted. For example, as the manipulation support information, an image as illustrated in FIG. 50 is displayed on the display device 320. For example, the shape of the large intestine, the bending of the insertion portion 203, a pushing amount of the large intestine by the insertion portion 203, or a force applied to the large intestine is displayed on the image. For example, as the support information associated with the manipulation, an image as illustrated in FIG. 51 is displayed on the display device 320. A direction in which the insertion portion 203 has to be inserted, a manipulation method for releasing the twist of the insertion portion 203, or the like is displayed on the image.

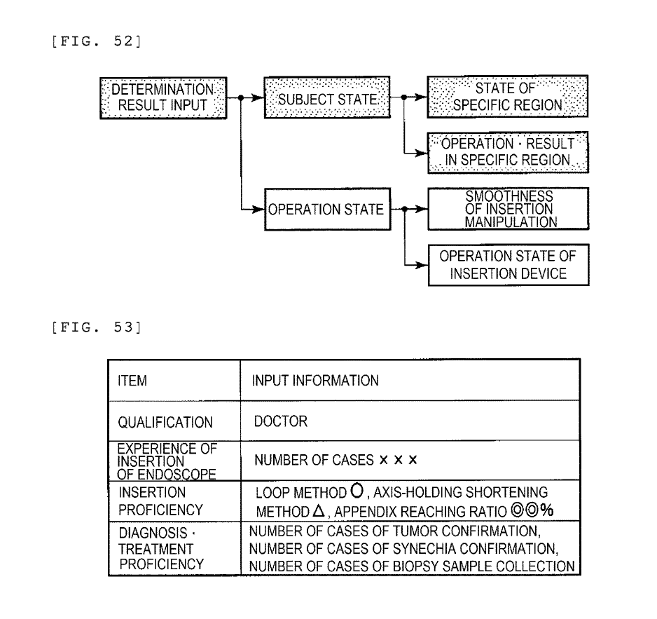

Other examples of the first manipulator information include determination of a state of the subject or the operation state, which is performed by the manipulator, an instruction to another person, or future response guidelines. FIG. 52 illustrates an example of the menu items in this case. In FIG. 52, a lightly shaded item is, for example, an item that has been selected. Here, “determination result input” for inputting determination results is selected, “subject state” is selected from “subject state” and “operation state” as the menu, “state of specific region” and “operation • result in specific region” are selected as the menu. Note that “smoothness of insertion manipulation” and “operation state of insertion device” are provided as the menu of “operation state”. Some of all of the input items may be automatically stored in the manipulation support information generating device 700. In addition, the automatically stored items may be configured to be appropriately set.

Examples of the second manipulator information that is input from the recording device 196 include the following information. An example of the second manipulator information includes user specific information. In other words, the second manipulator information can include information associated with experience of the user, a knowledge level of the user, a method or operative method that the user frequently uses. In addition, the second manipulator information can include information such as manipulation data during a past operation by the user or the provided support information.

FIG. 53 illustrates an example of the information. As illustrated in FIG. 53, the second manipulator information includes, a proficiency level of diagnosis • medical treatment, such as the qualification of the user as the doctor, for example, experience of the insertion of the endoscope in how many cases, for example, a proficiency level of the loop method, a proficiency level of the axis-holding shortening method, a proficiency level of the insertion as an appendix reaching ratio, the number of cases of tumor confirmation, the number of cases of synechia confirmation, or the number of cases of biopsy sample collection.

The information can be used to provide the manipulation instruction to the user, and can be used to generate the support information associated with the manipulation when the support information associated with the manipulation is generated with attention to an item with which a warning • abnormality was issued in the past.

In addition, an example of the second manipulator information includes the subject information. In other words, the second manipulator information can include age, gender, body data, vital information, medical history, examination/treatment history, or the like of the subject. In addition, the second manipulator information can include information such as manipulation data during a past operation that is received by the subject or the provided support information.

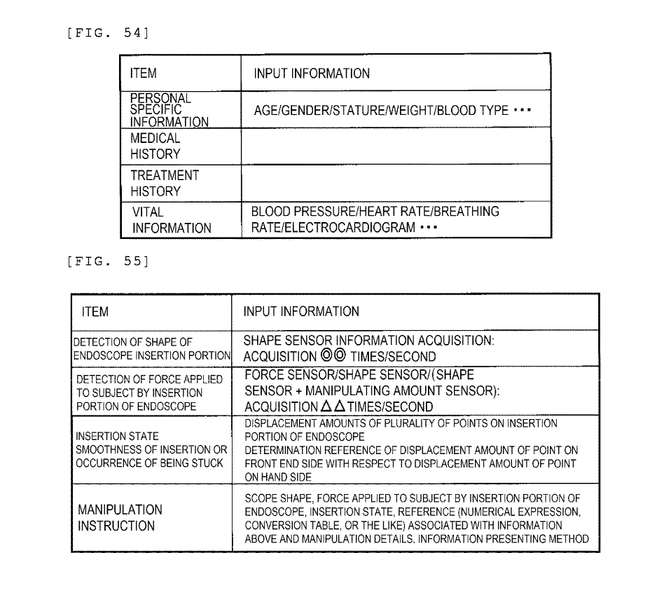

FIG. 54 illustrates an example of the information. As illustrated in FIG. 54, the second manipulator information includes personal specific information such as age, gender, stature, weight, a blood type, the medical history, treatment history, or vital information such as blood pressure, the heart rate, the breathing rate, or electrocardiogram.

The information can be used to provide the manipulation instruction to the user, and can be used in a case where manipulation, which was significantly different from the examination performed in the past, was performed, or when the manipulation support information is generated with attention to a spot having a warning or abnormality notified in the past

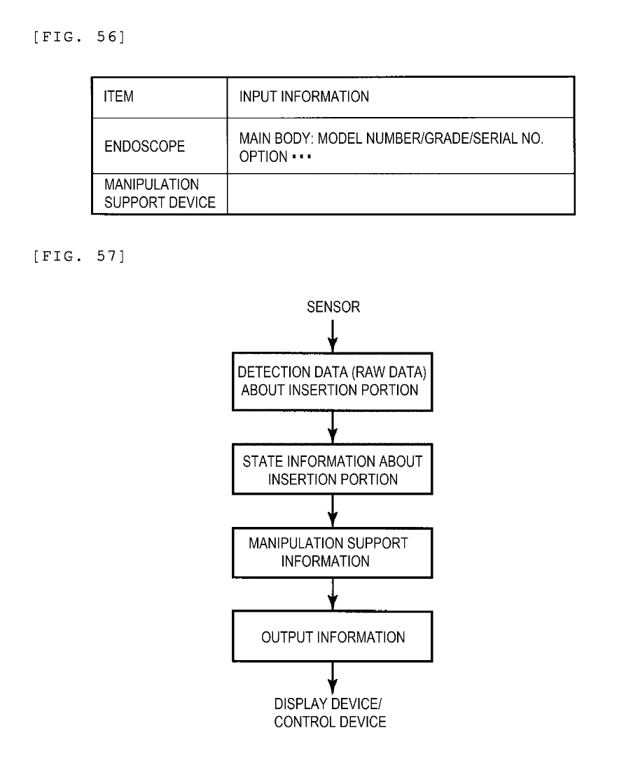

In addition, an example of the second manipulator information includes information associated with setting criteria. In other words, examples of the second manipulator information includes setting of a measuring instrument for generating the support information associated with the manipulation depending on a purpose of the examination or treatment, a data acquiring timing, the determination item, the determination criteria, or the like. FIG. 55 illustrates an example of the information.

As illustrated in FIG. 55, the second manipulator information includes, for example, setting information associated with shape detection of the endoscope insertion portion in which the information from the shape sensor is acquired several times per second. In addition, the second manipulator information includes setting information associated with detection of a force applied to the subject by the endoscope insertion portion in which the information is acquired from as sensor such as a force sensor, a shape sensor, and the shape sensor and a manipulating amount sensor, several times per second.

In addition, the second manipulator information includes information associated with smoothness of the insertion or an occurrence of being stuck (a deadlock state of the front end). In other words, the second manipulator information includes, for example, amounts of displacements of a plurality of points on the endoscope insertion portion, the amount of the displacement of the point on the front end side with respect to the amount of the displacement of the point on a hand side, or determination criteria. Based on the information described above, the information associated with the smoothness of the insertion or the occurrence of being stuck is generated as the manipulation support information.

In addition, the second manipulator information includes information associated with the manipulation instruction. In other words, the second manipulator information includes a scope shape, a force applied to the subject by the endoscope insertion portion, the insertion state, a criterion (a numerical expression, a conversion table, or the like) associated with the information above and the manipulation details, an information presenting method, or the like.

Based on the information described above, an amount of pushing/pulling of the endoscope 200, a direction or an amount of the twist, the manipulation of the bending portion, a posture change of the subject, an instruction of manipulation of air supply, air release, suction, or the like is generated as the support information associated with the manipulation. In addition, based on the information described above, a method of release from the loop of the insertion portion 203 and a method for shortening/straightening of a route are generated as the manipulation support information.

In addition, an example of the second manipulator information includes the device information. In other words, the second manipulator information includes specification of the used device (an endoscope, a measuring instrument, or the like), for example, a model number, a serial number, or a length of the endoscope 200, an installed measuring device, a mounted optional device, measurement content of the measuring device, a measurement range, detection accuracy, or the like. FIG. 56 illustrates an example of the information.

As illustrated in FIG. 56, the second manipulator information includes information associated with the endoscope 200 of a model number, a grade, or a serial number of the endoscope main body, or a model number, a grade, or a serial number of the optional device. In addition, the second manipulator information includes information as a model number, a grade, or a serial number of the insertion-extraction support device 100.

As described above, the use environment setting unit 730 performs the setting associated with the generation of the manipulation support information such that the support information associated with the manipulation which is necessary or is estimated to be necessary by the user is generated, based on the user-side information that is input to the user information determination unit 736.

The second manipulator information may be configured to be recorded in a recording medium such as a hard disk or a semiconductor memory, to be read, and to be appropriately updated.

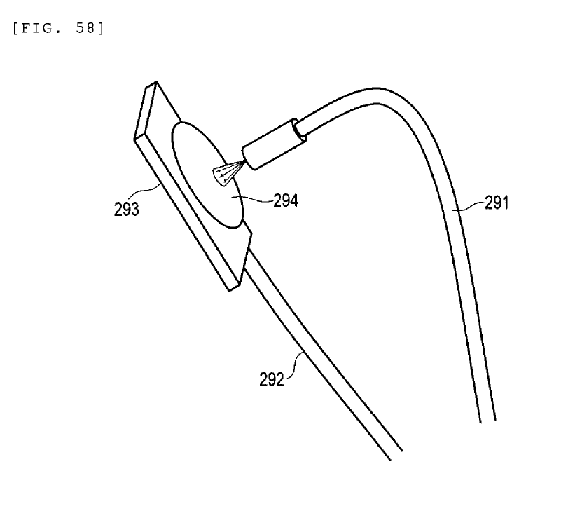

Next, an example of generating the support information associated with the manipulation that is performed in the manipulation support information generating unit 710. FIG. 57 illustrates an example of the information having the hierarchy. As illustrated in FIG. 57, the manipulation support information generating unit 710 acquires detection data as raw data associated with the insertion portion, from the sensor 201. The manipulation support information generating unit 710 acquires the state information associated with the insertion portion 203, based on the acquired detection data and the setting information acquired from the information generation setting unit 742. The manipulation support information generating unit 710 generates the support information associated with the manipulation, based on the acquired state information and the setting information acquired from the information generation setting unit 742. The manipulation support information generating unit 710 generates appropriate output information depending on an output target, based on the generated manipulation support information.

The output information is output to the display device 320 or the control device 310. The display device 320 displays the image, based on the input information. The image includes the support information associated with the manipulation. In addition, the control device 310 performs the feedback control, based on the output information. The control device 310 controls, for example, drive of an actuator 284 of a driving unit provided in the endoscope 200. Drive information to the actuator 284 includes, for example, information associated with an amount of the state of the insertion portion 203. The information includes, for example, information associated with drive of the actuator 284 such as an inserting-extracting amount of the insert, a twist amount, shape distribution, an amount of bending manipulation, distribution of vibration, distribution of temperature, distribution of hardness, or the like. As described above, the manipulation support information used in the feedback control is the information related to insertion manipulation support, risk avoidance, improvement of stability, or the like.

A part or the entirety of the manipulation support information generating device 700 including the manipulation support information generating unit 710 and the use environment setting unit 730 may be installed with an element disposed on a substrate, or may be integrated and may be installed as an integrated circuit. As described above, the manipulation support information generating unit 710 can be integrally installed with the use environment setting unit 730. Further, the storage unit is a non-volatile memory, and has a configuration in which stored content is updated. The storage unit may be integrally installed with the manipulation support information generating unit 710 and the use environment setting unit 730. In addition, a part or the entirety of the manipulation support information generating device 700 may be detachably mounted on the insertion-extraction support device 100. A part or the entirety of the manipulation support information generating device 700 is be detachably mounted on the insertion-extraction support device 100, and thereby it is possible to easily change the characteristics of the insertion-extraction support device 100, and the broad utility of the insertion-extraction support device 100 is improved.

Note that the insert, which is connected to the insertion-extraction support device 100 and of which the support information associated with the manipulation is generated by the insertion-extraction support device 100, is not limited to the endoscope 200. The insert that is connected to the insertion-extraction support device 100 may be a medical manipulator, a catheter, a medical and industrial endoscope, or the like. Such an insert can be configured to be used in observation or diagnosis of a subject, repair, modification, or treatment of the subject, and recording of the observation or diagnosis of the subject and the repair, modification, or treatment.

In addition, as illustrated in FIG. 58, the insertion-extraction support device 100 may be applied to a system in which a plurality of inserts is used. In other words, in an example illustrated in FIG. 58, a first insert 291 is configured to emit a laser beam from the front end thereof. In addition, a second insert 292 includes a light blocking plate 293 for laser processing. In a state in which the light blocking plate 293 is disposed on the rear side of a subject 294, the first insert 291 emits the laser, and thereby performs processing.

As described above, the first insert 291 and the second insert 292 are configured to perform in cooperation with each other. In addition, the first insert 291 and the second insert 292 may be configured to have different functions or performance from each other as illustrated in FIG. 58. In addition, at least one of the first insert 291 and the second insert 292 is used for observation or imaging. In other words, the first insert 291 and the second insert 292 may have an observation optical system. In addition, the first insert 291 and the second insert 292 have an imaging device and can be used for electronic observation. In addition, the first insert 291 and the second insert 292 have an imaging device and may be configured to be capable of recording image data.

In addition, the first insert 291 and the second insert 292 may have the same or equivalent function. The first insert 291 and the second insert 292 may be combined and may be configured to be capable of realizing one operational function.

In addition, the first insert 291 and the second insert 292 may have a configuration in which the first and second inserts are close to each other as illustrated in FIG. 58, or one insert is mounted in the other insert. The support information associated with the manipulation may be generated for one of the first insert 291 and the second insert 292 or for both. In addition, the support information associated with the manipulation may be generated for one insert, based on detection data of the other of the first insert 291 and the second insert 292.

Example of embodiments of the present invention relate to a manipulation support device. The manipulation support device comprises a primary information acquiring unit, a use environment setting unit and a manipulation support information generating unit.

The primary information acquiring unit can acquire detection data as primary information associated with a state of an insert from a sensor provided in the insert which is inserted into a subject.

The use environment setting unit can perform setting associated with generation of support information, based on at least one item of insert-side information associated with at least one of the insert and the sensor and user-side information associated with at least one of a manipulator who manipulates the insert or details of an operation performed by using the subject and the insert.

The manipulation support information generating unit can generate high-order information based on the setting, as the high-order information using information in hierarchies lower than the high-order information, which includes the primary information, thereby generating an information group having at least two hierarchies including the primary information, and generating the support information associated with the manipulation of the insert based on the information group.

The manipulation support information generating unit can generate the second-order or higher-order information, which is a part of the support information or required to generate the support information, based on the detection data, the first-order information, wherein the first-order information and the second-order or higher-order information comprise different order information groups.

The manipulation support information generating unit can generate the second-order information based on the detection data, the first-order information, and generating higher-order information, if any, based on lower-order information, wherein the second-order or higher-order information is a part of the support information or required to generate a part of the support information, and wherein the first-order information and the second-order or higher-order information comprise different order information groups.

The information group can include a plurality of items of different state information as items of information associated with states of different portions of the insert or as types of information having at least a different part, and the manipulation support information generating unit generates the support information based on the plurality of items of different state information.

The information groups comprise information regarding a plurality of different states of the inserted object, the information comprising at least one of information associated with states of different portions of the inserted object and information regarding different types of at least a portion of the inserted object; and wherein the support information for a manipulation of the inserted object based on the detection data and the setting information is generated based on the information regarding the different states of the inserted object.

The manipulation support information generating unit can generate, as the high-order information, the plurality of items of different state information associated with different positions of the insert in a longitudinal direction thereof.

The use environment setting unit can perform setting associated with at least one of generation details, a generation method, and a generation timing of the support information by the manipulation support information generating unit.

The manipulation support device can comprise a storage unit that stores at least one of the generation details, the generation method, and the generation timing of the support information.

The use environment setting unit can perform the setting associated with at least one of the generation details, the generation method, and the generation timing of the support information, based on the information stored in the storage unit.

The manipulation support device can comprise a storage unit that stores a setting criterion of at least one of the generation details, the generation method, and the generation timing of the support information.

The use environment setting unit can perform setting associated with at least one of the generation details, the generation method, and the generation timing of the support information, based on the setting criterion.

The use environment setting unit can perform determining of a use environment as an environment set when the insert is used, and setting associated with generation of the support information depending on the use environment.

The use environment setting unit can include at least one of an insert information determination unit that performs processing of the insert-side information and a user information determination unit that performs processing of the user-side information, and an information generation setting unit that performs the setting associated with the generation of the support information, based on at least one item of the insert-side information processed in the insert information determination unit and the user-side information processed in the user information determining portion.

The use environment setting unit can perform determining of the support information which is providable when the manipulation support device and the insert are combined and performs setting associated with the generation of the support information.

The manipulation support device can comprise an input unit that is configured to input information that specifies the support information which is requested by a manipulator.

The use environment setting unit can provide the providable support information to the manipulator.

The use environment setting unit can provide the support information other than the providable support information to the manipulator.

The use environment setting unit can perform, based on the user-side information, setting associated with the generation of the support information such that the manipulation support information generating unit generates the support information which is used by the manipulator or the support information which is estimated to be used by the manipulator.

The user-side information can be information associated with operation details performed by the manipulator.

The use environment setting unit can perform the setting associated with the generation of the support information such that the manipulation support information generating unit generates the support information related to the operation details.

The hierarchy can be based on a degree of processing of the detection data.

The manipulation support information generating unit and the use environment setting unit can be integrally installed.

The manipulation support information generating unit and the use environment setting unit can be integrated into one integrated circuit.

The manipulation support device can comprise a storage unit that has a configuration in which the manipulation support information generating unit and the use environment setting unit are integrally installed, and that is a non-volatile memory such that stored content is updated.

Example embodiments of the present invention relate to an insert system.

The insert system comprises the manipulation support device and the insert.

The manipulation support information generating unit and the use environment setting unit can be integrally installed.

The manipulation support information generating unit and the use environment setting unit can be detachably mounted on the manipulation support device.

The insert system can be configured to be used in observation or diagnosis of the subject, repair, modification, or treatment of the subject, and recording of the observation or diagnosis of the subject and the repair, modification, or treatment of the subject.

Example embodiments of the present invention relate to an insert system.

The insert system can comprise the manipulation support device, a first insert that functions as the insert, and a second insert that is configured to perform an operation in cooperation with the first insert.

The second insert can have a different function or performance from the first insert.

The second insert can be used in observation or imaging.

The second insert can have a function which is the same as or equivalent to that of the first insert.

The second insert can be combined with the first insert, thereby being capable of performing one operation function.

The first insert and the second insert can have a configuration in which the first and second inserts are close to each other or one insert is mounted in the other insert

The manipulation support device can generate the support information which is used in one of the first insert or the second insert, based on detection data of the other thereof.

Example embodiments of the present invention relate to a manipulation support method.

The method can comprise acquiring detection data as primary information associated with a state of an insert from a sensor provided in the insert which is inserted into a subject, performing setting associated with generation of support information, based on at least one item of insert-side information associated with at least one of the insert and the sensor or user-side information associated with at least one of a manipulator who manipulates the insert and details of an operation performed by using the subject and the insert, and generating high-order information based on the setting, as the high-order information using information in hierarchies lower than the high-order information, which includes the primary information, thereby generating an information group having at least two hierarchies including the primary information, and generating the support information associated with the manipulation of the insert based on the information group.

What is claimed is:

1. A manipulation support apparatus comprising:
a processor; and
memory storing instructions that when executed on the processor cause the processor to perform the operations of:
acquiring detection data from a sensor provided in an inserted object which is inserted into a subject body, the detection data being associated with a state of the inserted object;
deciding setting information based on at least one of:
inserted object information associated with at least one of the inserted object and the sensor; and
user information associated with at least one of a manipulator who manipulates the inserted object and an operation performed by using the subject body and the inserted object; and
generating support information for a manipulation of the inserted object based on the detection data and the setting information.

2. The manipulation support apparatus according to claim 1, wherein the detection data is a first order information, and
wherein generating support information comprises generating a higher order information based on the first order information, the higher order information comprising at least a second order information, the higher order information being a part of the support information or information that is required to generate a part of the support information, the first order information and the higher order information forming one or more information groups.

3. The manipulation support apparatus according to claim 1
wherein generating support information comprises generating the support information based on information regarding a plurality of different states of the inserted object, the information comprising at least one of information associated with states of different portions of the inserted object and information regarding different types of at least a portion of the inserted object.

4. The manipulation support apparatus according to claim 3,
wherein the memory further stores instructions that when executed on the processor cause the processor to perform the operation of:
generating information associated with different positions of the inserted object in a longitudinal direction thereof.

5. The manipulation support apparatus according to claim 1,
wherein deciding setting information comprises deciding the setting information associated with at least one of generation details, a generation method, and a generation timing of the support information.

6. The manipulation support apparatus according to claim 5, wherein the memory further stores information regarding at least one of the generation details, the generation method, and the generation timing of the support information,
wherein deciding setting information comprises deciding the setting information associated with at least one of the generation details, the generation method, and the generation timing of the support information, based on the stored information.

7. The manipulation support apparatus according to claim 5, wherein the memory further stores a setting criterion of at least one of the generation details, the generation method, and the generation timing of the support information,
wherein deciding setting information comprises deciding the setting information associated with at least one of the generation details, the generation method, and the generation timing of the support information, based on the setting criterion.

8. The manipulation support apparatus according to claim 1,
wherein the memory further stores instructions that when executed on the processor cause the processor to perform the operation of:
determining a use environment when the inserted object is used; and
wherein deciding setting information comprises deciding the setting information based on the use environment.

9. The manipulation support apparatus according to claim 8,
wherein the memory further stores instructions that when executed on the processor cause the processor to perform the operations of:
processing at least one of the inserted object information and the user information; and
deciding the setting information associated with the generation of the support information, based on at least one of the processed inserted object information and the processed user information.

10. The manipulation support apparatus according to claim 9,
wherein the memory further stores instructions that when executed on the processor cause the processor to perform the operation of:
determining providable support information based on the manipulation support device and the inserted object.

11. The manipulation support apparatus according to claim 10, further comprising:
an input device configured to receive input information that specifies the support information that is requested by the manipulator; and
wherein the memory further stores instructions that when executed on the processor cause the processor to perform the operations of:
providing the providable support information to the manipulator.

12. The manipulation support apparatus according to claim 11,
wherein the memory further stores instructions that when executed on the processor cause the processor to perform the operation of:
providing the support information other than the providable support information to the manipulator.

13. The manipulation support apparatus according to claim 9,
wherein deciding the setting information comprises deciding the setting information, based on the user information, associated with the generation of at least one of:
the support information which is necessary for the manipulator; or
the support information which is estimated to be necessary for the manipulator.

14. The manipulation support apparatus according to claim 13,
wherein the user information is information associated with the operation to be performed by the manipulator, and
wherein deciding the setting information comprises
deciding the setting information associated with the generation of the support information; and
generating the support information related to the operation.

15. An insert system comprising:
the manipulation support apparatus according to claim 1; and
the inserted object.

16. A manipulation support method comprising:
acquiring detection data from a sensor provided in an inserted object which is inserted into a subject body, the detection data being associated with a state of the inserted object;
deciding setting information, based on at least one of:
inserted object information associated with at least one of the inserted object and the sensor; and
user information associated with at least one of a manipulator who manipulates the inserted object and an operation performed by using the subject body and the inserted object; and
generating support information for a manipulation of the inserted object based on the detection data and the setting.

17. The manipulation support method according to claim 16,
wherein generating support information comprises generating a higher order information based on the first order information, the higher order information comprising at least a second order information, the higher order information being a part of the support information or information that is required to generate a part of the support information, the first order information and the higher order information forming one or more information groups.

18. The manipulation support method according to claim 16,
wherein generating support information comprises generating the support information based on information regarding a plurality of different states of the inserted object, the information comprising at least one of information associated with states of different portions of the inserted object and information regarding different types of at least a portion of the inserted object.

19. The manipulation support method according to claim 18, further comprising generating information associated with different positions of the inserted object in a longitudinal direction thereof.

20. The manipulation support method according to claim 16,
wherein deciding setting information comprises deciding the setting information associated with at least one of generation details, a generation method, and a generation timing of the support information.
